(19) United States
(12) Patent Application Publication
(10) Pub. No.: US 2014/0370533 A1
(43) Pub. Date: Dec. 18, 2014

(54) METHOD OF DETECTING ACTIVE TB

(71) Applicant: Norwegian Institute of Public Health, Oslo (NO)

(72) Inventors: Oistein Ihle, Oslo (NO); Terje Einar Michaelsen, Oslo (NO); Carol Joanne Church Holm-Hansen, Oslo (NO)

(73) Assignee: Norwegian Institute of Public Health, Oslo (NO)

(21) Appl. No.: 14/346,671

(22) PCT Filed: Sep. 19, 2012
(86) PCT No.: PCT/EP2012/068469

§ 371 Date: Mar. 21, 2014

(30) Foreign Application Priority Data
Sep. 21, 2011 (EP) 11182235.9

(51) Int. Cl. G01N 33/569; C07K 14/35; C07K 16/12

(52) U.S. Cl. CPC G01N 33/5695; C07K 16/1289; C07K 14/35; C07K 2317/52
USPC 435/7.94; 530/387.3; 530/395; 536/23.4; 536/23.7; 435/320.1; 435/252.33

(57) ABSTRACT

A method of detecting active TB in a patient comprising providing at least one peptide comprising an amino acid sequence with at least 80% sequence identity to a sequence selected from SEQ. ID NOS. 1 to 8, or an antigenic fragment thereof and contacting the peptide with a biological sample obtained from the patient. The presence of an antibody in the sample binding to the peptide is indicative of active TB in the patient.

### FIELD OF THE INVENTION

The present invention relates to a recombinant fusion protein and also to a nucleic acid molecule encoding the fusion protein and to a vector comprising the nucleic acid molecule. The present invention also relates to a kit for the detection of active tuberculosis in a patient. The invention, further, relates to a method of detecting active tuberculosis in a patient and to the use of a peptide as a biological marker for the presence of active tuberculosis in a patient.

### BACKGROUND OF THE INVENTION

Tuberculosis (hereinafter “TB”) is a serious infectious disease caused by certain strains of mycobacteria, principally Mycobacterium tuberculosis. It is estimated that one third of the world's population is infected with M. tuberculosis. However, about 90% of individuals infected with M. tuberculosis have a so-called “latent” infection (referred to as “Class 2” TB under the clinical classification system for TB) in which the bacteria lie inactive within macrophages in the individual. No symptoms of the infection are presented in an individual with a latent TB infection although a positive reaction is observed if a tuberculin skin test is carried out. Moreover, an individual with a latent TB infection cannot transmit TB to others. Nevertheless, 10% of individuals infected with M. tuberculosis develop “active” TB within their lifetime which involves symptoms such as cough, chest pains and hemoptysis. Active TB (referred to “Class 3” TB under the clinical classification system) will typically result in the death of the patient in around 50% of cases, if untreated, and it is individuals with active TB who are able to transmit the infection to others.

The prevalence of TB varies significantly from country to country. Broadly speaking, TB infection rates in developed countries are relatively low (e.g. in the United Kingdom, the national average was 15 cases per 100,000 in 2007) whereas rates of infection in developing countries are much higher (e.g. 1200 cases per 100,000 people in Swaziland in 2007). Detection and treatment of TB is essential to control the pandemic. Individuals with the active, untreated TB disease can infect 10-15 other people per year, according to some estimates. One of the difficulties in control of TB infection in developing countries is that there is often not ready access to a healthcare infrastructure which can reliably diagnose active TB infection in individuals quickly enough to prevent infected individuals from spreading TB more widely.

There is a range of different tests that can be carried out to detect active TB in a patient. A common test is a microbiological study in which a biological sample (e.g. sputum or pus) is obtained from a patient and investigated for evidence of M. tuberculosis. Typically, a sputum specimen is stained and examined microscopically for the presence of bacteria and thereafter subjected to culture. Sputum smear microscopy is not sensitive and culture, although reliable, takes six to eight weeks to complete and requires laboratory facilities that are not available in remote parts of developing countries.

Another common technique for the detection of TB is a tuberculin skin test. In brief, this test involves injecting tuberculin (a glycerol extract of M. tuberculosis) intradermally and then observing the patient 48 to 72 hours later. An individual who has been exposed to M. tuberculosis should display an immune response in the skin containing the tuberculin. The problem with the tuberculin skin test is that an individual with latent TB will also display a positive reaction to the skin test so the test does not enable individuals infected with active TB to be distinguished from those with a latent TB infection. Since a large proportion of individuals will test positive to this test, it is not feasible to isolate individuals on the basis of a positive result from the test.

There are also various other tests for TB infection which are known in the art. However, these other tests are generally characterised by yielding many false-negative results and not complying with WHO requirements. WHO has undertaken a laboratory-based evaluation of 19 commercially available rapid diagnostic tests for TB infection. The findings were published as part of a “Diagnostic Evaluation Series” in 2008. The performance, reproducibility and operational characteristics of the 19 commercially available tests was evaluated using 355 well-characterised archived serum samples from eight geographically diverse collection sites. Sera from TB-negative patients were used as a control population. A number of the tests analysed had a high specificity however, all of the tests with a high specificity (>95%) also had very low sensitivity (0.97-21%). In addition test performance was poor in patients with sputum smear-negative TB. Importantly, none of the tests analysed performed well enough to replace microscopy. Combining smear microscopy with the most rapid tests improved overall diagnostic sensitivity but yielded an unacceptable overall false positive rate of 42%. WHO concluded, based on this evaluation, that a simple to use, accurate, inexpensive and point of case diagnostic test for active TB is urgently needed.

As a result, although tuberculosis (TB) control programs have been successful in some countries, TB remains a major public health challenge worldwide. The lack of a rapid and widely accessible screening tool, that is easy to perform without laboratory facilities, is needed to curb the TB pandemic. Therefore, there is a need to provide a rapid screening tool for use in the field at the health post level to expedite same day referral, confirmatory testing and timely TB treatment. This will reduce diagnostic delay and thus the pool of infection below the critical reproductive rate for Mycobacterium tuberculosis.

Accordingly, the present invention seeks to alleviate one or more of the above problems. More specifically, the present invention seeks, in certain embodiments, to provide products and methods for the detection of active TB in individuals. The products and methods of the present invention arise from the surprising finding that individuals with active TB have a different repertoire of antibodies from individuals with latent TB. Individuals with active TB have antibodies specific for certain antigens of M. tuberculosis against which individuals with latent TB do not have antibodies. The products and methods of the invention are not intended to replace the microbiological tests for active TB described above. Instead, the products and methods of the present invention enable a simple means of rapidly detecting active TB in patients (or at least indicating a high likelihood of active TB in patients) using a technique which can distinguish between cases of active TB and latent TB and which, moreover, has a low rate of false-negative results. This test can be used at the health post level in the developing world. Since it is only individuals with active TB who are able to transmit the infection, the products and methods of the present invention enable infectious individuals to be identified and referred for confirmatory testing and treatment, thereby preventing the spread of TB infection within a population.

### SUMMARY OF THE INVENTION

According to a first aspect of the present invention, there is provided a method of detecting active TB in a patient comprising the steps of:
(a) providing at least one peptide comprising an amino acid sequence with at least 80% sequence identity to a sequence selected from SEQ. ID NOS. 1 to 8 or an antigenic fragment thereof;
(b) contacting the at least one peptide with a biological sample obtained from the patient; and
(c) detecting the binding of the at least one peptide with an antibody in the biological sample wherein the presence of an antibody binding to the peptide is indicative of active TB in the patient.

Conveniently, the at least one peptide is a plurality of peptides, each comprising a different sequence selected from the following (a) to (h):
(a) a sequence with at least 80% sequence identity to SEQ. ID NO. 1 or an antigenic fragment thereof;
(b) a sequence with at least 80% sequence identity to SEQ. ID NO. 2 or an antigenic fragment thereof;
(c) a sequence with at least 80% sequence identity to SEQ. ID NO. 3 or an antigenic fragment thereof;
(d) a sequence with at least 80% sequence identity to SEQ. ID NO. 4 or an antigenic fragment thereof;
(e) a sequence with at least 80% sequence identity to SEQ. ID NO. 5 or an antigenic fragment thereof;
(f) a sequence with at least 80% sequence identity to SEQ. ID NO. 6 or an antigenic fragment thereof;
(g) a sequence with at least 80% sequence identity to SEQ. ID NO. 7 or an antigenic fragment thereof; and
(h) a sequence with at least 80% sequence identity to SEQ. ID NO. 8 or an antigenic fragment thereof.

and wherein, in step (c), the presence of at least one antibody binding to at least one peptide is indicative of active TB in the patient.

Advantageously, the plurality of peptides comprises at least two of the following peptides:
(a) a sequence with at least 80% sequence identity to SEQ. ID NO. 1 or an antigenic fragment thereof;
(b) a sequence with at least 80% sequence identity to SEQ. ID NO. 2 or an antigenic fragment thereof; and
(c) a sequence with at least 80% sequence identity to SEQ. ID NO. 3 or an antigenic fragment thereof.

Preferably, the plurality of peptides consists of the following peptides:
(a) a sequence with at least 80% sequence identity to SEQ. ID NO. 1 or an antigenic fragment thereof;
(b) a sequence with at least 80% sequence identity to SEQ. ID NO. 2 or an antigenic fragment thereof; and
(c) a sequence with at least 80% sequence identity to SEQ. ID NO. 3 or an antigenic fragment thereof.

According to a second aspect of the present invention, there is provided the use of a peptide as a biological marker for the presence of active TB in a patient, when the peptide comprises an amino acid sequence with at least 80% sequence identity to a sequence selected from any of SEQ. ID NOS. 1 to 8 or an antigenic fragment thereof.

Preferably, the peptide comprises an amino acid sequence with at least 80% sequence identity to a sequence selected from any of SEQ. ID NOS. 1 to 3, or an antigenic fragment thereof.

According to a third aspect of the present invention, there is provided a method of treating active TB in a patient comprising detecting active TB in a patient in accordance with the second aspect of the present invention and administering to the patient a pharmaceutically effective amount of an active ingredient capable of treating TB.

Preferably, the active ingredient comprises an antibiotic.

According to a fourth aspect of the present invention, there is provided a kit for the detection of active TB in a patient comprising at least two different peptides selected from the following (a) to (h):
(a) a sequence with at least 80% sequence identity to SEQ. ID NO. 1 or an antigenic fragment thereof;
(b) a sequence with at least 80% sequence identity to SEQ. ID NO. 2 or an antigenic fragment thereof;
(c) a sequence with at least 80% sequence identity to SEQ. ID NO. 3 or an antigenic fragment thereof;
(d) a sequence with at least 80% sequence identity to SEQ. ID NO. 4 or an antigenic fragment thereof;
(e) a sequence with at least 80% sequence identity to SEQ. ID NO. 5 or an antigenic fragment thereof;
(f) a sequence with at least 80% sequence identity to SEQ. ID NO. 6 or an antigenic fragment thereof;
(g) a sequence with at least 80% sequence identity to SEQ. ID NO. 7 or an antigenic fragment thereof; and
(h) a sequence with at least 80% sequence identity to SEQ. ID NO. 8 or an antigenic fragment thereof.

Preferably, the at least two peptides are selected from the following (a) to (c):
(a) a sequence with at least 80% sequence identity to SEQ. ID NO. 1 or an antigenic fragment thereof;
(b) a sequence with at least 80% sequence identity to SEQ. ID NO. 2 or an antigenic fragment thereof;
(c) a sequence with at least 80% sequence identity to SEQ. ID NO. 3 or an antigenic fragment thereof.

Advantageously, the kit comprises all three peptides (a) to (c).

Conveniently, the kit comprises at least two recombinant fusion proteins according to the sixth aspect of the invention, wherein one of the at least two different peptides is the antigenic region in one of the at least two recombinant fusion proteins and the other of the at least two different peptides is the antigenic region in the other of the at least two recombinant fusion proteins.

According to a fifth aspect of the present invention, there is provided a kit for the detection of active TB in a patient comprising:
(a) a solid phase; and
(b) at least one peptide bound to the solid phase wherein the peptide comprises an amino acid sequence with at least 80% sequence identity to a sequence selected from any of SEQ. ID NOS. 1 to 8 or an antigenic fragment thereof.

Preferably, the solid phase comprises a substrate. Preferably the substrate is a membrane. Alternatively, the solid phase comprises a particle.

Advantageously, the kit comprises a plurality of particles each comprising at least one of the peptides bound thereto.

Preferably, the or each particle further comprises a detectable label.

Conveniently, the at least one peptide is a plurality of sets of different peptides and the peptide or peptides in each set comprises a different amino acid sequence selected from the following (a) to (h):
(a) a sequence with at least 80% sequence identity to SEQ. ID NO. 1 or an antigenic fragment thereof;
(b) a sequence with at least 80% sequence identity to SEQ. ID NO. 2 or an antigenic fragment thereof;
(c) a sequence with at least 80% sequence identity to SEQ. ID NO. 3 or an antigenic fragment thereof;
(d) a sequence with at least 80% sequence identity to SEQ. ID NO. 4 or an antigenic fragment thereof;
(e) a sequence with at least 80% sequence identity to SEQ. ID NO. 5 or an antigenic fragment thereof;
(f) a sequence with at least 80% sequence identity to SEQ. ID NO. 6 or an antigenic fragment thereof;
(g) a sequence with at least 80% sequence identity to SEQ. ID NO. 7 or an antigenic fragment thereof; and
(h) a sequence with at least 80% sequence identity to SEQ. ID NO. 8 or an antigenic fragment thereof.

wherein each particle has bound to it at least one of the peptides from one of the sets of peptides but does not have bound to it any peptides from any of the other sets of peptides.

More preferably, the set of different peptides consists of the following peptides:
(a) a sequence with at least 80% sequence identity to SEQ. ID NO. 1 or an antigenic fragment thereof;
(b) a sequence with at least 80% sequence identity to SEQ. ID NO. 2 or an antigenic fragment thereof; and
(c) a sequence with at least 80% sequence identity to SEQ. ID NO. 3 or an antigenic fragment thereof.

Preferably, the solid phase comprises a strip on which a fluid can pass by capillary action.

Advantageously, the kit further comprises a secondary antibody capable of binding human IgG.

Conveniently, the secondary antibody is bound to a detectable label or has a detectable label attachable to it

According to a sixth aspect of the present invention, there is provided a recombinant fusion protein comprising:
(a) at least one antigenic region comprising an amino acid sequence with at least 80% sequence identity to any of SEQ. ID NOS. 1 to 8 or an antigenic fragment thereof; and
(b) at least one anchoring region comprising a sequence of at least 25 amino acids.

Conveniently, the at least one antigenic region comprises a plurality of different antigenic regions having a different sequence selected from the following (a) to (h):
(a) a sequence with at least 80% sequence identity to SEQ. ID NO. 1 or an antigenic fragment thereof;
(b) a sequence with at least 80% sequence identity to SEQ. ID NO. 2 or an antigenic fragment thereof;
(c) a sequence with at least 80% sequence identity to SEQ. ID NO. 3 or an antigenic fragment thereof;
(d) a sequence with at least 80% sequence identity to SEQ. ID NO. 4 or an antigenic fragment thereof;
(e) a sequence with at least 80% sequence identity to SEQ. ID NO. 5 or an antigenic fragment thereof;
(f) a sequence with at least 80% sequence identity to SEQ. ID NO. 6 or an antigenic fragment thereof;
(g) a sequence with at least 80% sequence identity to SEQ. ID NO. 7 or an antigenic fragment thereof; and
(h) a sequence with at least 80% sequence identity to SEQ. ID NO. 8 or an antigenic fragment thereof.

Preferably, at least one antigenic region is N-terminal to the at least one anchoring region.

Advantageously, at least one antigenic region is C-terminal to the at least one anchoring region.

More preferably, the at least one antigenic region having a sequence selected from (a), (b), (d) and (g) is C-terminal to the at least one anchoring region. Preferably, the at least one antigenic region having a sequence selected from (a), (b), (c), (e), (f) and (h) is N-terminal to the at least one anchoring region.

Preferably, the fusion protein comprises a plurality of anchoring regions.

Conveniently, the at least one anchoring region comprises at least constant domains 2 and 3 and a hinge region of an immunoglobulin Fc region, preferably a murine IgG2a Fc region or a corresponding rabbit, rat or sheep Fc region. Preferably, the at least one anchoring region comprises an entire IgG molecule, more preferably a murine IgG2a antibody. Alternatively, the at least one anchoring region comprises a histidine (His) tag or a Glutathione S-transferase (GST) tag. Conveniently, the at least one anchoring region comprises a plurality of anchoring regions.

Advantageously, the fusion protein is glycosylated. Suitably, the fusion protein has a prokaryotic glycosylation pattern. Alternatively, the fusion protein has a eukaryotic glycosylation pattern.

Preferably, the glycosylation is selected from the group comprising N-linked glycosylation, O-linked glycosylation, P-linked glycosylation and C-linked glycosylation.

According to a seventh aspect of the present invention, there is provided a nucleic acid molecule comprising a nucleotide sequence encoding a recombinant fusion protein according to the first aspect of the invention or the antigenic region according to the tenth aspect of the invention.

According to a eighth aspect of the present invention, there is provided a vector comprising a nucleic acid molecule according to the second aspect of the invention or the antigenic region according to the tenth aspect of the invention.

According to a ninth aspect of the present invention, there is provided a host cell comprising a nucleic acid molecule according to the seventh aspect of the invention or a vector according to the eighth aspect of the invention and capable of expressing a recombinant fusion protein according to the sixth aspect of the invention or the antigenic region according to the tenth aspect of the invention.

According to a tenth aspect of the present invention, there is provided an antigenic region comprising an amino acid sequence with at least 80% sequence identity to any of SEQ ID NOS. 1 to 8, or an antigenic fragment thereof, wherein the antigenic region is glycosylated. Suitably, the fusion protein has a prokaryotic glycosylation pattern. Alternatively, the fusion protein has a eukaryotic glycosylation pattern. Preferably, the glycosylation is selected from the group comprising N-linked glycosylation, O-linked glycosylation, P-linked glycosylation and C-linked glycosylation.

The term “active TB” as used herein refers to a tuberculosis infection (whether pulmonary or otherwise) that is clinically active. In particular “active TB” is a tuberculosis infection that is within Class 3 of the World Health Organisation's official classification system. “A method of detecting active TB” as used herein refers to a method of distinguishing between “active TB” and “latent TB” or between a sample having “active TB” and a sample wherein no TB is present.

The term “amino acid” as used herein refers to naturally occurring and synthetic amino acids, as well as amino acid analogues and amino acid mimetics that have a function that is similar to the naturally occurring amino acids. Naturally occurring amino acids are those encoded by the genetic code, as well as those modified after translation in cells (e.g. hydroxyproline, gamma-carboxyglutamate, and O-phosphoserine). The phrase “amino acid analogue” refers to compounds that have the same basic chemical structure (an alpha carbon bound to a hydrogen, a carboxy group, an amino group, and an R group) as a naturally occurring amino acid but have a modified R group or modified backbones (e.g. homoserine, norleucine, methionine sulfoxide, methionine methyl sulphonium). The phrase “amino acid mimetic” refers to chemical compounds that have different structures.

The term “antigenic fragment” as used herein in relation to an amino acid sequence refers to a polypeptide having a contiguous series of amino acid residues present in the amino acid sequence, which polypeptide comprises an epitope that can be bound by an antibody. As such, antigenic fragments may comprise at least 6, 8, 10, 12 or 15 amino acid residues.

The term “nucleotide” as used herein refers to naturally occurring nucleotides and synthetic nucleotide analogues that are recognised by cellular enzymes.

The terms “polynucleotides”, and “nucleic acid molecules” are used interchangeably herein to refer to a polymer of multiple nucleotides. The nucleic acid molecules may comprise naturally occurring nucleic acids or may comprise artificial nucleic acids such as peptide nucleic acids, morpholin and locked nucleic acids as well as glycol nucleic acids and threose nucleic acids.

The terms “polypeptide”, “peptide” and “protein” are used interchangeably herein to refer to a polymer of amino acid residues. The terms apply to amino acid polymers in which one or more amino acid residues is a modified residue, or a non-naturally occurring residue, such as an artificial chemical mimetic of a corresponding naturally occurring amino acid, as well as to naturally occurring amino acid polymers.

The term “recombinant” as used herein refers to a nucleic acid molecule or a polypeptide which is located in a non-naturally occurring context and which has been produced by artificial intervention. For example, a first polypeptide isolated from other polypeptides or linked by a peptide bond to a second polypeptide sequence having a different amino acid sequence from any polypeptide with which the first polypeptide is associated in nature is a recombinant polypeptide.

The term “glycosylation” as used herein refers to a co-translation or post-translation modification whereby glycans are attached to proteins or other molecules. Proteins produced and secreted by eukaryotic cells have a different pattern of glycosylation than proteins produced and secreted by prokaryotic cells. There are several different types of glycans that can be attached, namely N-linked glycans, O-linked glycans, Phospho-linked glycans or C-linked glycans.

The term “prokaryotic glycosylation pattern” as used herein, refers to the glycans that are attached to a protein when the protein is produced and secreted by a prokaryotic cell.

The term “eukaryotic glycosylation pattern” as used herein, refers to the glycans that are attached to a protein when the protein is produced and secreted by a eukaryotic cell.

In this specification, the percentage “identity” between two sequences is determined using the BLASTP algorithm version 2.2.2 (Altschul, Stephen F., Thomas L. Madden, Alejandro A. Schäffer, Jinghui Zhang, Zheng Zhang, Webb Miller, and David J. Lipman (1997), “Gapped BLAST and PSI-BLAST: a new generation of protein database search programs”, Nucleic Acids Res. 25:3389-3402) using default parameters. In particular, the BLAST algorithm can be accessed on the internet using the URL http://www.ncbi.nlm.nih.gov/blast/.

### BRIEF DESCRIPTION OF THE FIGURES

FIG. 1 is a schematic diagram of a fusion protein in accordance with one embodiment of the present invention.

FIG. 2 is a schematic diagram of a fusion protein in accordance with another embodiment of the present invention.

FIG. 3 is a schematic diagram of a fusion protein in accordance with a further embodiment of the present invention.

FIG. 4 is a schematic diagram of a fusion protein in accordance with a further embodiment of the present invention.

FIG. 5 is a schematic diagram of the components of an ELISA protocol in accordance with one embodiment of the present invention. FIG. 5(b) shows a kit in accordance with one embodiment of the invention and FIG. 5(a) shows a component thereof. FIGS. 5(c) to 5(e) show the use of the kit.

FIG. 6 is a schematic diagram of a laminar flow immunoassay in accordance with another embodiment of the present invention. FIG. 6(a) is a plan of the laminar flow immunoassay of FIG. 6(b) is a side elevation thereof.

FIG. 7 is a schematic diagram of the components of the labelled particle assay in accordance with a further embodiment of the present invention. FIG. 7(a) shows a kit in accordance with this embodiment of the invention. FIG. 7(a) shows a kit in accordance with this embodiment of the invention. FIGS. 7(b) and 7(c) show the components of the embodiment in use.

FIG. 8 is a graphical representation of the Bio-Plex Assay results. FIG. 8(a) illustrates the number of patients testing positive for active TB in a TB-confirmed Ethiopian test group and in a healthy HIV-negative Ethiopian community control group. FIG. 8(b) illustrates the number of patients testing positive for active TB in a TB-confirmed Tanzanian test group and in a healthy HIV-negative Tanzanian community control group. FIG. 8(c) illustrates the number of individuals testing positive for active TB among Ethiopian patients suspected of having TB (but confirmed non-TB with conventional tests), in a TB-confirmed Ethiopian test group, in a healthy HIV-negative Ethiopian community control group, and among Norwegian TB-negative controls.

FIG. 9 is a graphical representation of the Rapid Test TB “prototype” ELISA results illustrating the number of patients testing positive for active TB in a TB-confirmed Ethiopian test group and in a healthy HIV-negative Ethiopian community control group.

FIG. 10 is a graphical representation of the Bio-Plex Assay results, illustrating the number of patients testing positive for active TB using Rv0934 fusion protein in a TB-confirmed Ethiopian test group and in a healthy HIV-negative Ethiopian community control group.

FIG. 11 is a graphical representation of the Bio-Plex Assay results illustrating the number of patients testing positive for active TB using Rv1759 fusion protein in a TB-confirmed Ethiopian test group and in a healthy HIV-negative Ethiopian community control group.

FIG. 12 is a graphical representation of the Bio-Plex Assay results, illustrating the number of patients testing positive for active TB using Rv1886 fusion protein in a TB-confirmed Ethiopian test group and in a healthy HIV-negative Ethiopian community control group.

FIG. 13 is a graphical representation of the Bio-Plex Assay results, illustrating the number of patients testing positive for active TB using Rv3874 fusion protein in a TB-confirmed Ethiopian test group and in a healthy HIV-negative Ethiopian community control group.

FIG. 14 is a graphical representation of the Bio-Plex Assay results, illustrating the number of patients testing positive for active TB using Rv3881 c fusion protein in a TB-confirmed Ethiopian test group and in a healthy HIV-negative Ethiopian community control group.

FIG. 15 is a graphical representation of the Bio-Plex Assay results, as illustrated in FIG. 8(a), showing the signal contribution for each fusion protein used in the test.

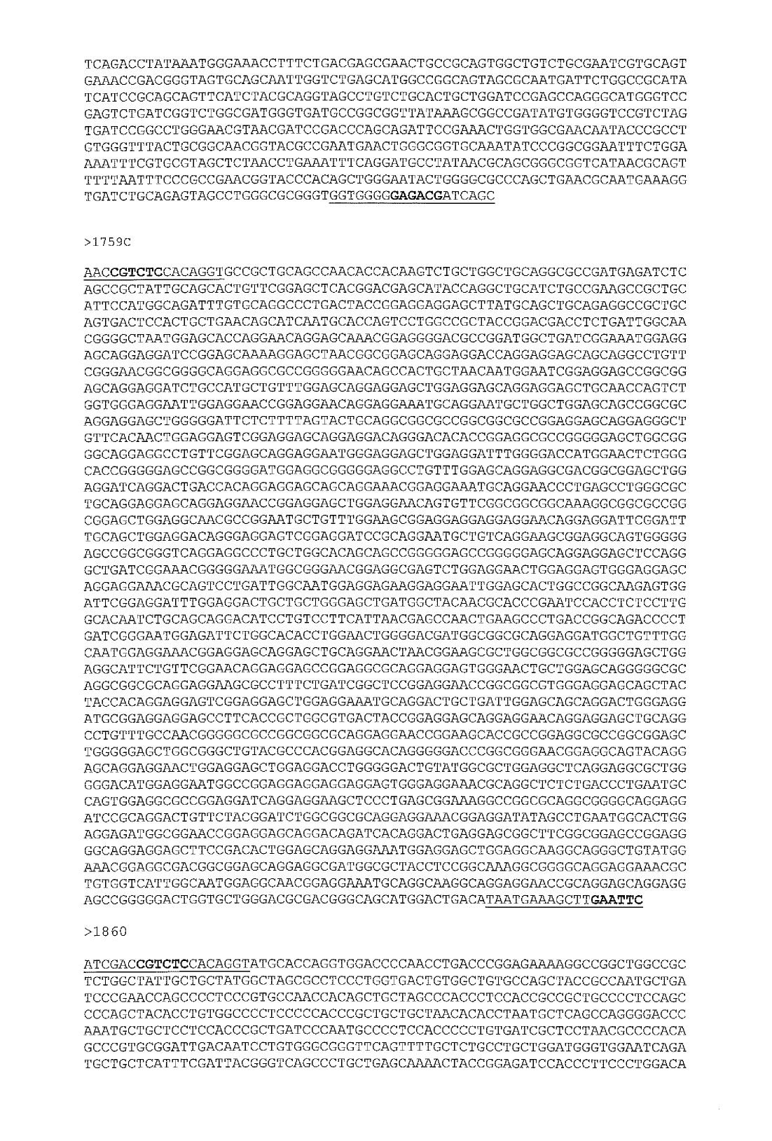

FIG. 16 illustrates the eight optimised DNA sequences for each antigenic region, containing the coding sequence of the antigen (no underline), the flanking sequences (underlined) and the restriction enzyme sites (in bold).

FIG. 17 is a graphical representation of the Bio-Plex Assay results, illustrating the number of patients testing positive for active TB (S+C+H−, i.e sputum smear-positive by microscopy, mycobacterial culture-positive and HIV-negative) using the antigen mix comprising Rv3881c, Rv0934 and Rv1886, in an active TB confirmed Vietnamese test group and in a healthy HIV-negative Vietnamese community control group (CC). MFI (Median Fluorescence Intensity) value of 7000 yields 83.8% sensitivity and 96.6% specificity.

### DETAILED DESCRIPTION OF THE INVENTION

The present invention is based on the finding that individuals with an active TB infection have circulating antibodies which are specific for certain protein antigens of the M. tuberculosis organism hereinafter referred to as “target antigens”. The target antigens are all proteins encoded by genes within the M. tuberculosis genome. Importantly, individuals with latent TB do not, generally, have circulating antibodies against these antigenic proteins. The target antigens of the invention and the names of the genes encoding them are set out in Table 1.

TABLE 1
The target antigens of the invention

| | | | Optimized DNA |
|---|---|---|---|
| | Alternative names for | Protein sequence | coding sequence |
| Gene | Gene | (SEQ. ID NO.) | (SEQ. ID NO.) |
| Rv3881c | — | 1 | 9 |
| Rv0934 | PSTS1; PBP-1 | 2 | 11 |
| Rv1886c | fbpβ | 3 | 13 |
| Rv3875 | | 8 | 23 |
| Rv1759c | — | 4 | 15 |
| Rv3874 | | 7 | 21 |
| Rv1860 | | 6 | 19 |
| Rv1980c | MPT64 | 5 | 17 |

The preferred orientation of the antigenic regions in relation to the anchoring region of the fusion protein is presented in Table 2.

TABLE 2
Preferred orientation of the antigenic regions
in relation to anchoring region

| | | | Preferred |
|---|---|---|---|
| Gene | C-terminal | N-terminal | Orientation |
| Rv3881c | Yes | Yes | C-terminal |
| Rv0934 | Yes | Yes | N-terminal |
| Rv1886c | No | Yes | |
| Rv1759c | Yes | No | |
| Rv1980c | No | Yes | |
| Rv1860 | No | Yes | |
| Rv3874 | Yes | No | |
| Rv3875 | No | Yes | |

The invention provides, in one aspect, a recombinant fusion protein comprising an antigenic region and an anchoring region. Since the fusion protein is recombinant, the anchoring region is of a sequence that is not found adjacent to the antigenic region in nature. The antigenic region comprises a target antigen, this is to say an amino acid sequence encoded by Rv3881c, Rv0934, Rv1886c, Rv1759c, Rv1980c, Rv1860, Rv3874 and Rv3875. However, the antigenic region need not contain an entire target antigen. Instead, the antigenic region may comprise a shorter amino acid sequence, which is still antigenic.

Furthermore, the amino acid sequence of the antigenic region may be different from the target antigen sequence that is encoded by the corresponding M. tuberculosis gene, provided that it has at least 80% sequence identity to the naturally occurring target antigen sequence and is bound by antibodies against the naturally occurring target. In other embodiments, the amino acid sequence of the antigenic region has at least 90%, 95% or 99% sequence identity to the target region. It is also preferred that any addition or substitution of the amino acid sequence set out in SEQ. ID NOS. 1 to 8 results in the conservation of the properties of the original amino acid side chain. That is to say the substitution or modification is “conservative”.

Conservative substitution tables providing functionally similar amino acids are well known in the art. Examples of properties of amino acid side chains are hydrophobic amino acids (A, I, L, M, F, P, W, Y, V), hydrophilic amino acids (R, D, N, C, E, Q, G, H, K, S, T), and side chains having the following functional groups or characteristics in common: an aliphatic side-chain (G, A, V, L, I, P); a hydroxyl group containing side chain (S, T, Y); a sulphur atom containing side-chain (C, M); a carboxylic acid and amide containing side-chain (D, N, E, Q); a base containing side-chain (R, K, H); and an aromatic containing side-chain (H, F, Y, W). In addition, the following eight groups each contain amino acids that are conservative substitutions for one another (see e.g. Creighton, Proteins (1984):

1) Alanine (A), Glycine (G);

2) Aspartic acid (D), Glutamic acid (E);

3) Aspargine (N), Glutamine (Q);

4) Arginine (R), Lysine (K);

5) Isoleucine (I), Leucine (L), Methionine (M), Valine (V);

6) Phenylalanine (F), Tyrosine (Y), Tryptophan (W);

7) Serine (S), Threonine (T); and

8) Cysteine (C), Methionine (M).

In some embodiments, the antigenic region comprises non-naturally occurring amino acids but is still bound by antibodies against one of the target antigens.

The anchoring region comprises a sequence of at least 25 amino acids in length and is adjacent to the antigenic region. In alternative embodiments the anchoring region is larger than 25 amino acids and may, for example, comprise at least 30, 40, 50, 60, 70, 80, 90, 100 or 150 amino acids. In preferred embodiments, the anchoring region comprises at least a part of the Fc region of an immunoglobulin molecule, in particular constant domains 2 and 3 and the hinge region thereof in the case of an Fc region from an IgG, IgA or IgD antibody or constant domains 2 to 4 and the hinge region thereof in the case of an Fc region from an IgE or IgM antibody. Such Fc regions have the ability to bind Protein A and Protein G which enables straightforward purification of the fusion protein. The Fc region can come from any species with mouse, rat and rabbit being preferred. A particularly preferred anchoring region is the murine IgG2a Fc region as there are polyclonal antibodies available which are exceptionally selective for this Fc region which enables a high level of sensitivity in the assays described below. In an alternative embodiment the anchoring region is an entire IgG molecule, preferably a murine IgG2a antibody. An alternative anchoring region is a His-tag or glutathione S-transferase. The purpose of the anchoring region is to increase the solubility of the fusion protein and to increase the stability of the fusion protein. In some embodiments, the anchoring region also enables binding of the fusion protein to another component (e.g. a substrate or an antibody) whilst allowing the antigenic region to be displayed, without blocking binding of an antibody to the antigenic region.

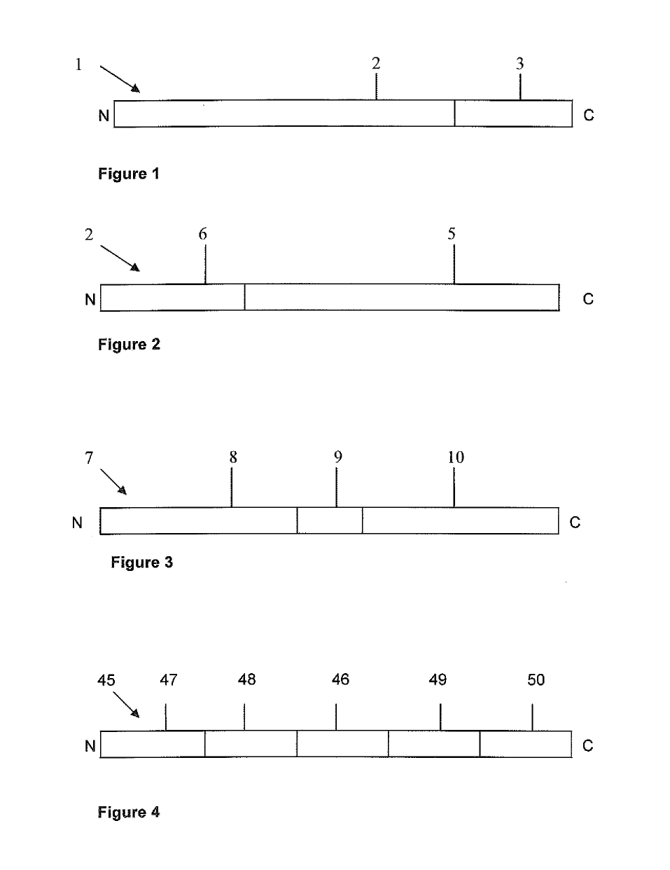

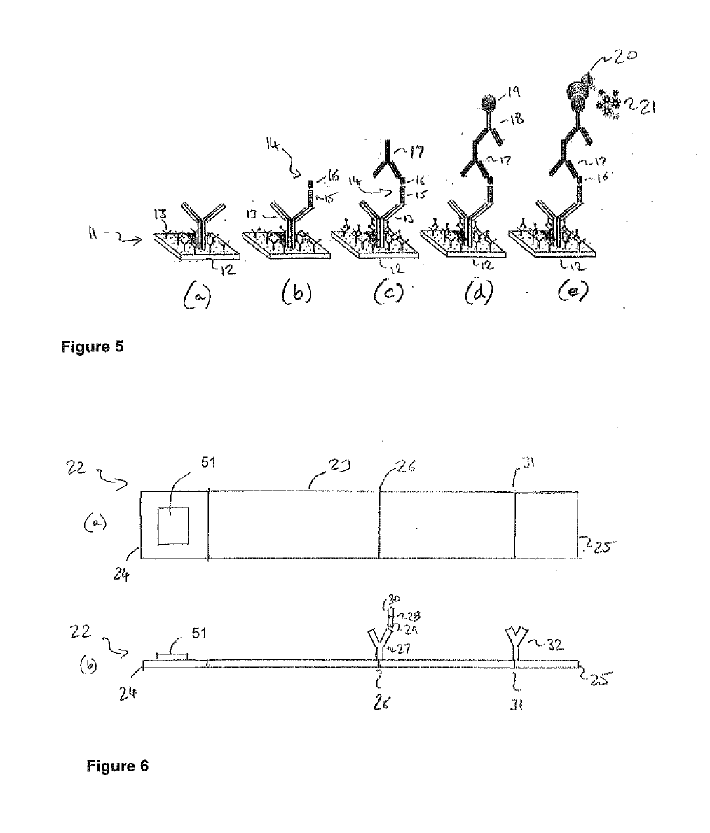

Referring, now, to FIG. 1, a recombinant fusion protein 1, in accordance with one embodiment of the present invention is shown. The recombinant fusion protein comprises an antigenic region 2 which is at the N-terminal end of the fusion protein with an anchoring region 3 C-terminal thereof. Referring to FIG. 2, an alternative embodiment of the present invention is shown in which a fusion protein 4 comprises an antigenic region 5 which is at the C-terminal end of the fusion protein with an anchoring region 6 being N-terminal of the antigenic region 5.

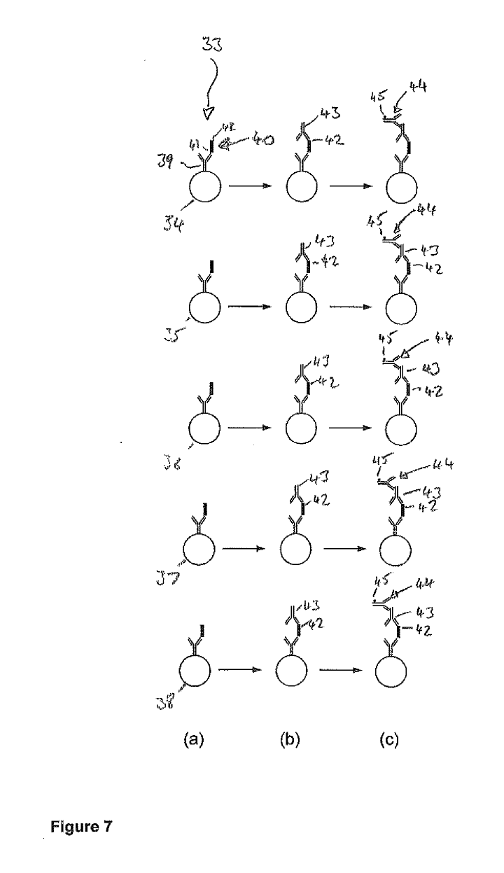

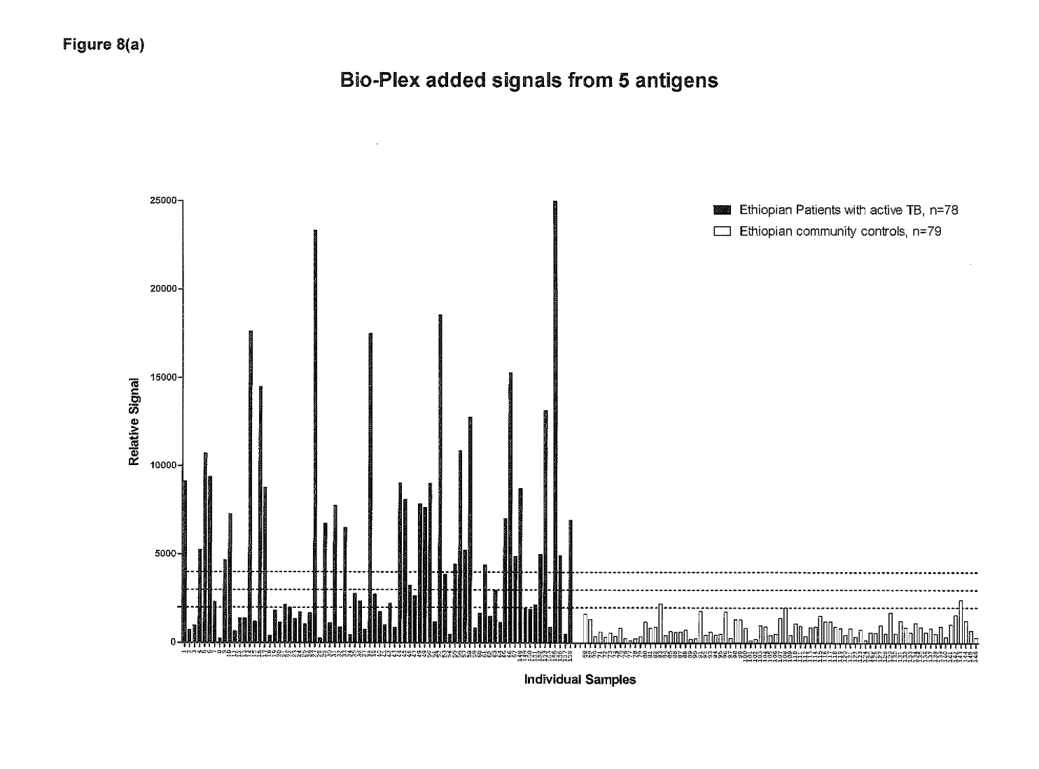

In still further embodiments of the present invention, there are provided fusion proteins having a plurality of antigenic regions. For example, in one embodiment, shown in FIG. 3, a fusion protein 7 comprises a first antigenic region 8, at its N-terminus. At the C-terminal end of the first antigenic region 8, there is provided an anchoring region 9. At the C-terminal end of the anchoring region 9, there is provided a second antigenic region 10. Thus, in this embodiment, the fusion protein 7 comprises an anchoring region 9 which is flanked by first and second antigenic regions 8, 10. Referring to FIG. 4, an alternative embodiment of the present invention is shown in which, a fusion protein 45, comprises, at its N-terminus a first antigenic region 47, and a second antigenic region 48, and at its C-terminus, a third antigenic region 49, and a fourth antigenic region 50. An anchoring region 46 is flanked by the first and second antigenic regions 47 and 48 and the third and fourth antigenic regions 49 and 50.

In another aspect of the present invention, there are provided nucleic acid molecules which encode a fusion protein of the present invention. Further provided are nucleic acid molecules which encode the antigenic regions of the present invention. Owing to the degeneracy of the nucleotide code, a wide range of different sequences may encode the same amino acid sequence. The nucleic acid molecule may be of any nucleotide sequence and may even comprise nucleic acid analogues, provided that the nucleic acid molecule encodes the fusion protein. Nevertheless, it is preferred that the nucleic acid molecule is of a sequence which is optimised for expression in a particular host cell (e.g. mammalian cells). Nucleotide sequences optimised for expression in human cells and encoding target antigens of the invention are listed in Table 1.

In some embodiments, the nucleic acid molecule encodes additional sequences, aside from those which specifically encode the fusion protein or the antigenic region. For example, in some embodiments, the nucleic acid molecule also encodes a 5′ terminal signal peptide for excretion of the fusion protein by a host cell in which it is expressed. For example if the host cell is a mammalian host cell then it is preferred that the signal peptide be a mammalian leader sequence. An exemplary signal peptide is Human Spd195, having the sequence of SEQ ID NO. 25. A further exemplary signal peptide is a signal peptide having the sequence of SEQ ID NO. 26. The signal peptide may be one which directs the fusion protein to one of several internal cell compartments, such as the nucleus, golgi apparatus or the mitochondria. In a preferred embodiment the signal peptide is one which directs the fusion protein to be secreted out of the cell entirely. Other additional sequences that the nucleic acid molecule may comprise include regulatory regions such as enhancers.

In another aspect of the present invention, there is provided a vector comprising a nucleic acid molecule according to the present invention. In particular embodiments, the vector may, in addition to the sequence of the nucleic acid molecule, comprise components such as an origin of replication, genetic markers, antibiotic resistance genes, COS sites, cloning sites and targeting sequences. Exemplary vectors include cosmids, and viral vectors such as bacteriophage.

In a further aspect of the present invention, there is provided a host cell comprising a vector of the present invention or a nucleic acid molecule of the present invention recombinantly introduced into the genome of the host cell. In preferred embodiments, the host cell is a mammalian cell such as a 293E cell. The host cell is capable of expressing the nucleotide sequence encoding the fusion protein.

In order to synthesize a fusion protein in accordance with the present invention, a nucleic acid molecule of the present invention is first synthesised using standard microbiological techniques. The nucleic acid molecule is then incorporated into an expression vector, comprising the necessary elements for sustaining and reproducing the vector in a host cell and also elements, such as an antibiotic resistance gene, for identifying transfected cells. A suitable expression vector is the pFRIDA vector. The pFRIDA vector may be a pFRIDA-IgG2a-N having a sequence of SEQ ID NO. 27. Alternatively, the pFRIDA vector may be a pFRIDA-IgG2a-C having a sequence of SEQ ID NO. 28. A host cell is then transfected with the expression vector using techniques known in the art such as the calcium phosphate technique, electroporation, Polyethylenimine (PEI) transfection or by using a cationic lipid. Preferred host cells are mammalian cells, in particular 293E cells. After confirming that the host cell has been transfected with the vector of the invention, the cell is cultured and the nucleic acid molecule encoding the fusion protein is expressed.

In some embodiments, the nucleic acid molecule also encodes a secretory signal peptide and the anchoring region is an Fc region from an IgG molecule. In these embodiments, the fusion protein is translated containing the secretory signal peptide which leads to the protein being exuded from the cell (and the signal peptide cleaved off) by virtue of the host cell's expression systems. In particular, the Fc region enables the fusion protein to exit the plasma membrane of the cell by improving the solubility and stability of the antigenic region. In these embodiments, the fusion protein can then be recovered from the extracellular culture medium in a relatively straightforward manner by, for example, purification on a column (e.g. a protein A column if the anchoring region is an Fc region).

However, in other embodiments, where the secretory signal peptide is absent and/or the anchoring region does not comprise an Fc region, the expressed fusion protein remains intracellular and is recovered by lysing the host cell and purifying the fusion protein, again, by passing the protein through a column containing binding elements (e.g. antibodies) capable of binding the anchoring region.

Once a fusion protein of the invention has been expressed and purified, it is, in certain embodiments of the invention, incorporated into a kit for the detection of active TB in a patient.

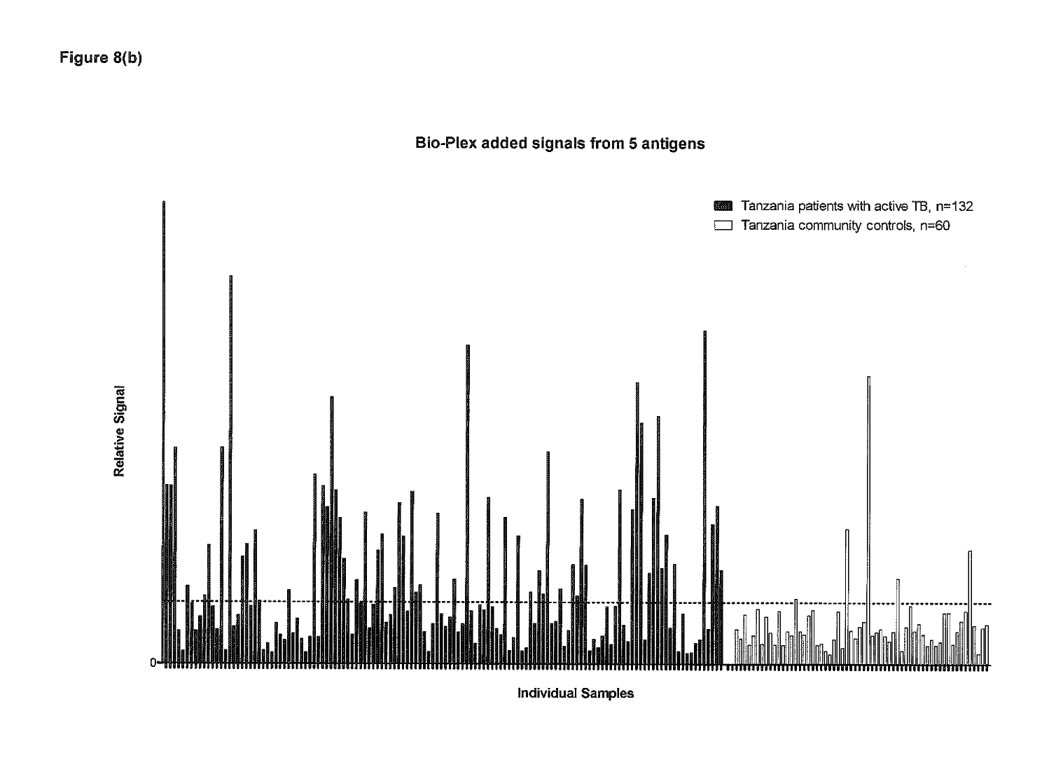

Referring to FIGS. 5(a) to 5(e), one embodiment of a kit is shown schematically. A kit 11 (of which only a portion is shown in FIG. 5) comprises an ELISA plate 12 (a section of which is shown in FIGS. 5(a) to 4(e)). Immobilised on the surface of the ELISA plate 12 are a plurality of goat anti-murine IgG2a antibodies 13 via covalent bonds. Each of the anti-murine IgG2a antibodies 13 is specific for the murine IgG2a hinge/Fc region which forms the anchoring region of the fusion protein in this embodiment.

FIG. 5(b) disclose a section of the prepared kit 11 in which a fusion protein 14 has been added to the surface of the ELISA plate. The fusion protein 14 comprises an anchoring region 15 bound to the variable domain of an anti-murine IgG2a and an antigenic region 16 which is displayed. The kit 11 also comprises a supply of a secondary antibody (18) (not shown in FIG. 5(b)) which is an anti-human IgG antibody conjugated to a reporter enzyme 19. The kit 11 is also provided with a supply of a substrate of the enzyme which undergoes a colour change on conversion by the reporter enzyme. The kit 11 of which a section is shown in FIG. 5(b) is in the form in which it is distributed and sold.

In use of the kit 11 for detection of active TB, an individual presents to a clinic for testing. The individual has symptoms which suggest possible infection with TB. Alternatively, the individual may be tested because he or she has had contact with another individual who has been infected with TB or may present as part of a general screening programme. A biological sample is then taken from the individual. In this embodiment, the biological sample is a blood sample but in other embodiments, a sample of bodily fluid such as saliva is obtained instead. Serum, if necessary, is obtained from the blood sample by centrifugation. Whole blood samples are suitable for use in rapid point of care assays. The biological sample is then prepared (e.g. by addition of a protease inhibitor). The prepared sample is then deposited on the ELISA plate 12.

If the individual has active TB then the individual's blood/serum contains an active TB-specific antibody 17 which binds the antigenic region 16 of the fusion protein 14 as is shown in FIG. 5(c).

Referring to FIG. 5(d), the next stage in the use of the kit is shown. The secondary antibody 18 to which is conjugated the reporter enzyme 19 is then added to the ELISA plate 12. The secondary antibody 18 binds to the Fc region of the active TB-specific antibody 17. Subsequently, the plate 12 is washed in order to remove unbound antibody and the enzyme substrate 20 is deposited on the plate 12, as is shown in FIG. 5(e). The substrate 20 is converted by the reporter enzyme in order to produce a detectable product 21. Thus the presence of the detectable product 21 is indicative of the presence of the active TB-specific antibody 17 in the biological sample of the individual, which is, in turn, indicative of the individual having active TB.

In the case of an individual without active TB, the individual's biological sample does not contain antibodies capable of binding the antigenic region 16. Thus, no antibody binds to the fusion protein 14 that is deposited on the ELISA plate 12. The secondary antibody 18 is washed from the plate before addition of the enzyme substrate 20 and, in the absence of the reporter enzyme 19, none of the detectable product 21 is produced. Thus for a patient who does not have active TB, none of the detectable product 21 is produced.

The kit 11 thus permits rapid detection of active TB in an individual, or at least a high likelihood of active TB in an individual. An individual identified in this way will be referred for confirmatory testing at a clinic or hospital with laboratory facilities. An individual with positive confirmatory test results will be treated with appropriate antibiotics such as isoniazid or rifampicin and thus the spread of infection will be reduced. The kit 11 has a low level of false-negative results. To exclude false-positive results, and ensure correct diagnosis, individuals testing positive in an assay based on the kit 11 will be re-tested with conventional techniques such as sputum smear microscopy and mycobacterial culture.

It is to be understood that many variations in the kit 11 described in this embodiment may be made. For example, in some embodiments, the anti-immune IgG2a antibodies 13 are not provided and, instead, the fusion protein 14 is directly bound to the plate 12, for example by a covalent bond. In these embodiments, it is not essential that the anchoring region be provided since, in some embodiments, the antigenic region 16 is directly bound to the plate 12 (e.g. by a covalent bond). In still further embodiments, the antigenic region 16 is attached to the plate 12 by virtue of a binding pair other than an antigen-antibody. For example, in one embodiment, the plate 12 is coated with biotin and the antigenic region 16 is bound to streptavidin or avidin in place of the anchoring region. Thus on depositing the antigenic region 16 on the plate, the streptavidin or avidin binds to the biotin and the antigenic region 16 is immobilised on the plate 12.

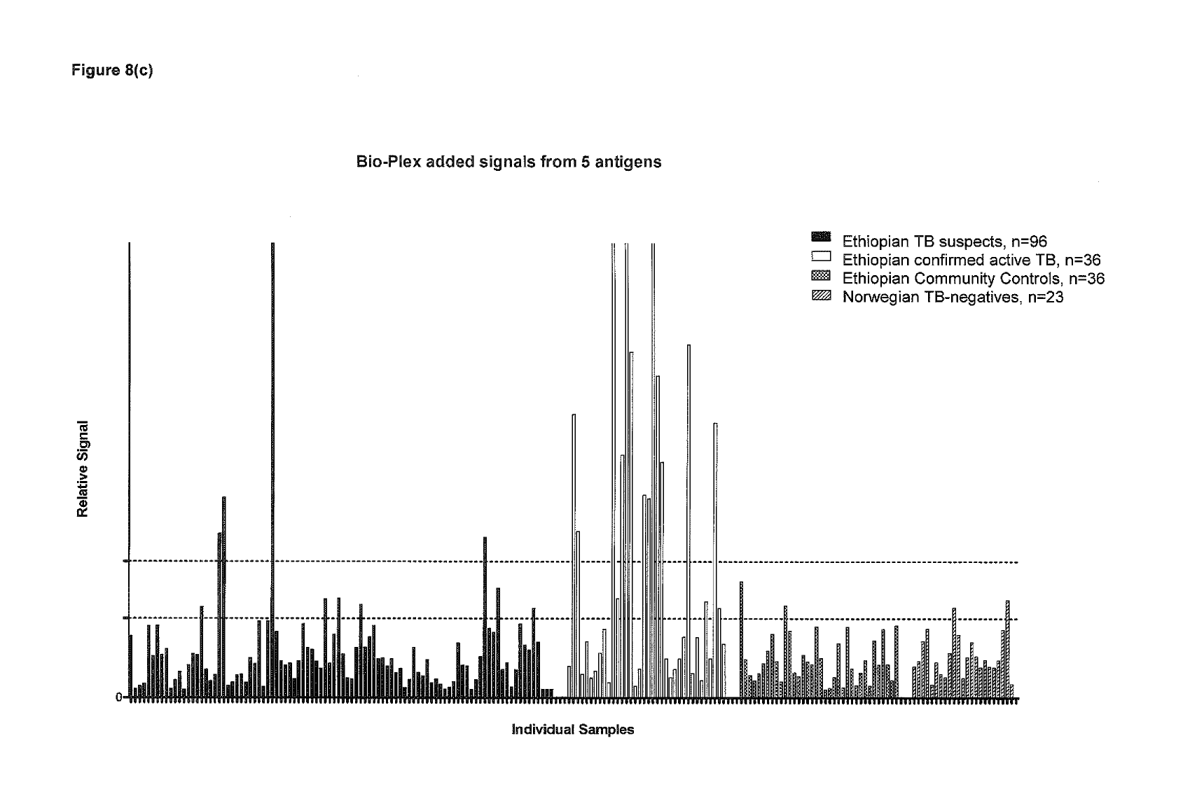

Referring to FIGS. 6(a) and 6(b), a kit 22 for the detection of active TB in a patient in accordance with another embodiment of the present invention is shown. The kit 22 comprises a nitrocellulose strip 23 having first and second ends 24, 25. The strip is such that fluid may pass along it via capillary action. Adjacent to the first end 24 of the strip 23 is provided a sample receiving zone 51 comprising an absorbent pad. Approximately half way between the first and second ends 24 and 25 of the strip 23 is provided a detection zone 26. The detection zone 26 comprises a plurality of goat anti-murine IgG2a antibodies immobilised in a line which is perpendicular to the longitudinal axis of the strip 23. The goat antibodies 27 are immobilised on the strip 23 in the same manner as described in relation to the embodiment shown in FIG. 6. Bound to the variable region of the goat antibodies 27 is provided a fusion protein 28 which consists of an anchoring region 29 which is bound to the goat antibody 27 and an antigenic region 30 which comprises one of the target antigens as described above.

Adjacent to the second end 25 of the strip 23 is provided a control zone 31, which comprises a plurality of anti-human IgG antibodies 32 immobilised covalently on the strip 23 in a line perpendicular to the longitudinal axis of the strip 23.

Also provided with the kit 22 is a supply of a secondary antibody (not shown) which is also an anti-human IgG antibody and which is conjugated to a detectable label such as a latex particle or a gold particle.

In use of the kit as is shown in FIG. 6, a biological sample is obtained from an individual, as described in the previous embodiments. If the sample has been correctly obtained then it contains antibodies from the individual and, if the individual has active TB, these antibodies include active TB-specific antibodies. The biological sample is added to phosphate buffered saline (PBS) or Chaser Buffer. The sample may be treated with a number of compounds, for example, a protease inhibitor can be added to avoid degradation of antibodies, detergents can be added to minimize unspecific binding in the test, blocking agents can be added such as BSA (bovine serum albumine) and skimmed milk to enhance the specific binding in the test or thiocyanate salts can be added in order to abolish low avidity antibody-antigen interactions, eliminating background signal in the test.

The sample is deposited on the sample receiving zone 51. The sample soaks through the absorbent pad and, by capillary action, passes from the first end 24 to the second end 25 of the strip 23. If the individual has active TB then, when the biological sample reaches the detection zone 26, active TB-specific antibodies in the biological sample bind to the antigenic region 30 of the fusion protein 28 and are immobilised there. The remainder of the biological sample, including other antibodies in the sample that are not specific for TB, passes to the second end 25 of the kit 22. The non-TB-specific antibodies in the sample bind to the anti-human IgG antibodies 32 in the control zone 31 and are immobilised there.

Subsequently, a buffer containing the secondary antibody (not shown) is added to the sample receiving zone 51. Upon it being deposited at the sample receiving zone 51, the secondary antibody soaks through the absorbent pad and passes along the strip 23 from the first end 24 to the second end 25, by capillary action. When the secondary antibody reaches the detection zone 26, it binds to the active TB-specific antibody from the individual and the concentration of the detectable label is such that a visible line appears at the detection zone 26. The appearance of this visible line at the detection zone 26 is thus indicative of the presence of antibodies capable of binding a target antigen in the individual and thus is indicative of the individual having active TB.

An excess of secondary antibody is provided on the sample receiving zone 51 so some of the secondary antibody passes through the detection zone 26 and binds to the non-TB-specific antibodies that are immobilised at the control zone 31. The concentration of the detectable label at the control zone 31 is thus indicative that the assay has completed because the biological sample has passed through the detection zone 26 and the outcome of the assay should be visible at the detection zone 26. Indeed, the relative intensities of the lines at the detection zone 26 and the control zone 31 can be compared to determine whether a visible line is present at the detection zone 26. This helps to avoid false negative results which could occur if, for example, there is insufficient liquid in the biological sample for any of the biological sample to reach the detection zone 26 or if the biological sample has been incorrectly obtained and does not contain any antibodies from the individual.

In cases where the individual does not have active TB, the biological sample does not have active TB-specific antibodies capable of binding a target antigen. After depositing of the biological sample on the sample receiving zone 51, the sample passes along the strip 23, as described above, but passes through the detection zone 26 since there are no antibodies in the biological sample capable of binding the antigenic region 30 of the fusion protein 28. Thus the sample passes all the way to the second end 25 of the strip 23. The non-TB-specific antibodies in the sample bind to the anti-human IgG antibodies 32 in the control zone 31 and are immobilised there. When the secondary antibody is deposited on the sample receiving zone 51, this too passes along the strip 23 from the first end 24 to the second end 25, as described above but passes through the detection zone 26 because there is no active TB-specific antibody immobilised at the detection zone 26 for the secondary antibody to bind to. However, the secondary antibody does bind to the non-TB-specific antibodies that are immobilised at the control zone 31 and thus the secondary antibody is immobilised at the control zone 31 and the concentration of the detectable label at the control zone 31 is visible to the naked eye. The presence of a visible line at the control zone 31 is indicative that the assay preformed using the kit 22 has run its course and the absence of a visible line at the detection zone 26 is indicative that the individual does not have active TB.

It is to be understood that in other embodiments of the present invention, the goat antibody 27 is not provided. Instead, in these embodiments, the fusion protein 28 is immobilised directly onto the surface of the strip 23, for example by covalent binding. Indeed, in some of these embodiments, it is not even necessary for a fusion protein to be provided since the antigenic region 16 is covalently bound directly to the surface of the strip 23. Furthermore, the provision of the control zone 31 is not essential to the invention since the assay can be run without confirmation that it has been completed.

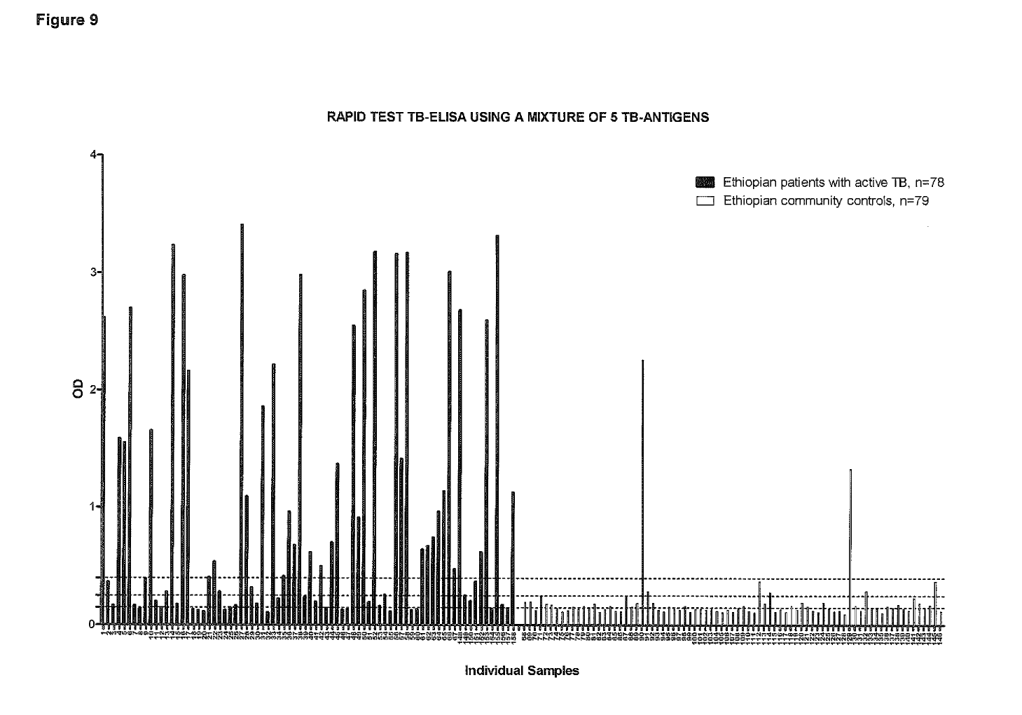

Referring to FIG. 7, another embodiment of a kit for the detection of active TB in an individual is shown in schematic form. As shown in FIG. 7(a), the kit 33 comprises five sets of polymer particles 34 to 38 (for convenience, each set of particles is shown as a single particle in FIG. 7). Each set of particles 34 to 38 is marked with a different label, such as a series of dyes selected from the Alexa Fluor family of fluorescent dyes produced by Molecular Probes. Referring now to the particle 34 of the first set, the particle 34 has bound to its surface a plurality of goat anti-murine IgG2a antibodies 39, for example by protein G affinity coupling. The variable region of the goat antibody 39 is bound to a fusion protein 40 which comprises an anchoring region 41 which is bound to the goat antibody 39 and an antigenic region 42 which has the sequence of SEQ. ID NO. 1.

Each of the second to fifth sets of particles 35 to 38 also has displayed on it a goat antibody and a fusion protein. However, for each set of particles 34 to 38 the fusion protein 40 is different in that the antigenic region is of a different sequence. More specifically, while the antigenic region 42 of the first set of particles 34 comprises the sequence of SEQ. ID NO. 1, the second antigenic region 42 of the fusion protein 40 on the second set of particles 35 comprises the sequence of SEQ. ID NO. 2; the third set of particles 36 has an antigenic region 42 comprising the sequence of SEQ. ID NO. 3 and so forth up the fifth set of particles 38 which display an antigenic region 42 comprising the sequence of SEQ. ID NO. 5. Thus the kit 33 comprises a mixture of these five sets of particles 34 to 38. The kit also comprises a supply of a secondary antibody 44 which is an anti-human IgG antibody carrying a detectable label 45 such as a fluorescent or luminescent dye. In some alternative embodiments, the secondary antibody does not carry the detectable label, itself. Instead, the detectable label is attachable to the secondary antibody, for example by a binding pair such as biotin and streptavidin or avidin.

In use, a biological sample is obtained from an individual and is processed in the manner described for the previous embodiments. The processed sample is then mixed with the five sets of particles 34 to 38. If the individual has active TB and thus has active TB-specific antibodies in the biological sample which bind to the target antigens then the individual's antibodies 43 bind to the respective antigenic regions 42 displayed on the corresponding first to fifth sets of particles 34 to 38, as shown in FIG. 7(b).

After mixing of the biological sample with the set of particles, the secondary antibody 44 carrying the detectable label 45 is mixed with the particles. The secondary antibody 44 binds the Fc region of all of the antibodies in the biological sample, irrespective of the binding specificity of the human antibodies, as is shown in FIG. 7(c). The mixture of particles 34 to 38 is then analysed using a flow cytometer. More specifically, the flow cytometer examines each particle and detects the presence or absence of the detectable label 45 in association with each particle, as well as the identity of the dye which marks the particle, itself. Since each set of particles 34 to 38 displays a different antigenic region 42, the output from the flow cytometer is therefore indicative of whether the biological sample contained antibodies capable of binding each of the antigenic regions 42. Thus this embodiment allows a multiplex detection for antibodies specific for five different antigenic regions 42 representative of the different target antigens.

If, however, the individual from whom the biological sample is taken does not have active TB then the biological sample does not contain any active TB-specific antibodies. Accordingly none of the individual's antibodies in the biological sample bind to the antigenic regions 42 displayed by the five sets of particles 34 to 38. Thus when the secondary antibody 45 is added, although it binds to the human antibodies 44 in the mixture, it is not associated with the particles 34 to 38, themselves, since there is no link between the secondary antibody 44 and the particles 34 to 38. When the particles 34 to 38 are analysed in the flow cytometer, there is no association between the detectable marker 45 and each particle 34 to 38. The absence of such a signal is therefore indicative of the absence of active TB in the individual.

It is also to be understood that in some circumstances an individual from whom the biological sample is taken has antibodies that bind some of the target antigens, but not all of the target antigens. For example, in one embodiment, the individual has antibodies capable of binding epitopes in SEQ. ID NOS. 1 to 3 but not epitopes in SEQ. ID NOS. 4 or 5. In these circumstances, the secondary antibody becomes associated with only the first to third sets of particles 34 to 36 but does not become associated with the fourth and fifth sets of particles 37, 38 since there is no active TB-specific antibody to bind the secondary antibody 44 to the antigenic region 42 of the fourth and fifth sets of particles 37, 38. In these circumstances, the flow cytometer detects the detectable label 45 only in association with the first to third sets of beads 34 to 36 and generates a signal indicative of this. This information then enables clinicians to make a decision as to the diagnosis of the individual. For example, in one embodiment the detection in a biological sample from an individual of active TB-specific antibodies against at least one of the target antigens (i.e. SEQ. ID NOS. 1 to 8) is indicative of the individual having active TB. In a preferred embodiment, the detection in a biological sample from an individual of active TB-specific antibodies against at least one of the target antigens, selected from SEQ ID No. 1 to 3, is indicative of the individual having active TB. In a still preferred embodiment, detection of active TB-specific antibodies against three target antigens, SEQ ID No. 1 to 3, is indicative of the individual having active TB.

It is to be appreciated that in some embodiments the goat antibody 39 is not provided and instead the anchoring regions 41 are bound directly to the surface of their respective particles 34 to 38. Alternatively, the anchoring regions 41 are omitted and the antigenic regions 42 are bound directly to the surface of their respective particles 34 to 38. The anchoring regions 41 or antigenic regions 42 may be bound to the surface of their respective particles 34 to 38 either by a covalent bond or by another binding pair such as biotin and streptavidin or avidin.

It is also to be understood that the present invention is not limited to the provision of kits comprising five different sets of particles. It is to be understood that in a further embodiment eight different sets of particles are provided, in which for each set of particles the fusion protein is different in that the antigenic region is of a different sequence, namely SEQ. ID NO. 1 to SEQ ID NO. 8, respectively. In some embodiments 1, 2, 3, 4, 6, or 7 different sets of particles are provided instead. In a preferred embodiment, 3 different sets of particles are provided.

Although the above described method utilises fusion proteins, it is to be appreciated that in some embodiments, the anchoring region of the fusion protein is not present and that the method may be carried out using only the antigenic region of the protein. In this approach, the method is carried out using at least one protein having a sequence selected from SEQ ID NO. 1 to SEQ ID NO. 8. In a preferred embodiment, at least one protein, having a sequence selected from SEQ ID NO. 1 to SEQ ID NO. 3, is provided. In a different embodiment, three proteins are provided, each protein having a different sequence selected from SEQ ID NO. 1, SEQ ID NO. 2 and SEQ ID NO. 3.

### EXAMPLES

### Materials and Methods

Cloning and Production of the TB Fusion Protein

Synthesis of TB Antigen

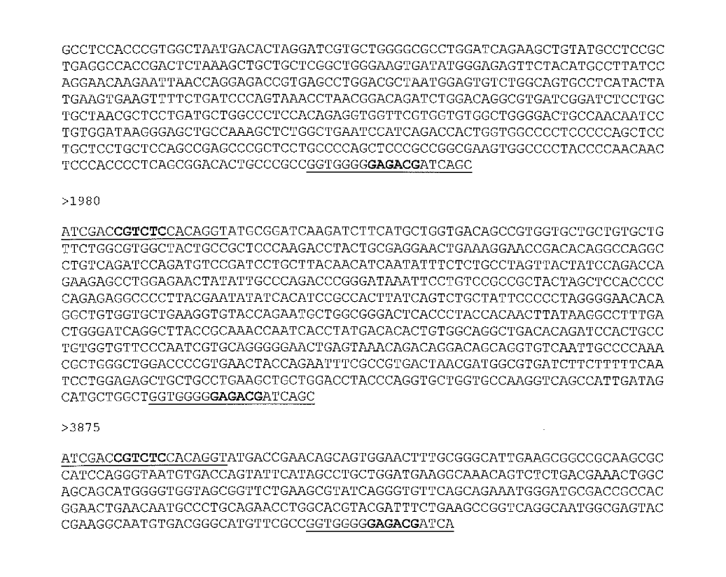

The sequence encoding the gene of the TB antigen to be produced as a fusion protein was retrieved from publicly available databases. Flanking sequences containing endonuclease restriction sites were added to the gene sequence, to make the gene compatible with expression in the pFRIDA vector. The flanking sequences containing the restriction enzyme sites added to each gene sequence are shown in FIG. 16. The genes were synthesised by GenScript (USA), optimizing the DNA codons for expression in human cells. The synthesised genes where received as an insert in a pUC57 vector construct.

Cloning of TB Antigen

2 μg of the pUC57 vector containing the synthesized gene encoding the TB antigen was digested with restriction enzymes, Esp3I (www.fermentas.com) and EcoRI (www.fermentas.com) in a reaction volume of 60 μl for C-terminal cloning. For N-terminal cloning, only the Esp3I enzyme was added to the reaction. The digestion reaction was incubated for 60 minutes at 37° C. The resulting DNA fragments were separated by agarose gel electrophoresis, and the DNA fragment corresponding to the gene encoding the TB-antigen was isolated by excision. The excised gel fragment was treated according to the protocol of the Qiagen (www.qiagen.com) MinElute Gel Extraction Kit, and the DNA was recovered.

The recovered DNA encoding the TB antigen was added to the corresponding pFRIDA vector for either N-terminal or C-terminal expression as a fusion protein. The pFRIDA vector had been previously digested with either Esp3I, or Esp3I and EcoRI, and then purified by agarose gel electrophoresis. A molar ratio of 3:1 of the insert:vector was mixed, and 0.5 μl T4-DNA ligase (www.neb.com) was added in a total reaction of 25 μl. The ligation reaction was incubated at 20-24° C. for 1-2 hours.

Transformation of E. coli

30 μl of competent XL-10 Gold E. coli cells where added 10 μl of the ligation reaction and treated as described by the manufacturer (www.agilent.com). The resulting transformation mix was spread on solid agar plates containing 100 μg/ml ampicillin.

The agar plates were incubated 12-16 hours at 37° C.

Bacteria Colony Isolation, Growth and DNA Plasmid Isolation

From the incubated agar plates, 5-10 single colonies where isolated and grown individually in 2 ml of liquid 2×YT medium (www.sigma-adrich.com) containing 100 μg/ml ampicillin (www.sigma-adrich.com). The colonies where incubated for 6-8 hours with shaking (300 rpm) at 37° C. Each of the colony suspensions were then treated with the QIAprep Spin Miniprep Kit, according to the manufacturer's (www.qiagen.com) instructions. This resulted in the recovery of about 10 μg of plasmid DNA.

Verification of DNA Plasmid

To verify that the gene encoding the TB antigen had been ligated successfully into the pFRIDA vector, the isolated plasmids where digested with restriction enzymes NheI and EcoRI, and the resulting pattern of DNA fragments analyzed on an agarose gel. In some cases standard DNA sequencing was performed.

PEI Transfection of 293E Cells

Plasmids verified to contain the gene encoding the TB antigen were used in a PEI transfection procedure.

Approximately 2 million 293E cells were suspended in 10 ml of DMEM (www.invitrogen.com) with glutamine and 10% fetal calf serum (FCS) was added to a 15 ml bottle (Nunc, Denmark) and incubated overnight in a CO₂-incubator.

150 μl RPMI medium (www.invitrogen.com) was then added to 3 μg plasmid DNA and heated to 80° C. for 5 minutes, and then cooled to 0-4° C. 25 μl of PH solution (www.polysciences.com) was then added and the reaction mix was incubated at room temperature for 8-12 minutes. 1350 μl of DMEM (www.invitrogen.com) with glutamine and 10% FCS (www.invitrogen.com), was then added.

The cell growth medium in the 15 ml Nunc (www.nunc.com) bottle was removed, and the transformation reaction (about 1.5 ml) was added to the bottle, ensuring the liquid covered all the cells in the bottle. The bottle was then incubated for 2 hours in the CO₂-incubator, before adding 10 ml of DMEM with glutamine and 10% FCS.

Isolation of TB Fusion Antigen Containing Growth Media

The growth media contained approximately 1-5 μg/ml of the TB antigen murine IgG2a fusion protein after 2-5 days growth of the transfected 293E cells. 10 ml of growth media was removed after 4-5 days, and replaced with 10 ml of fresh media for a possible second harvest of TB fusion protein in 5-10 days. The collected growth media was centrifuged at 2-4000×g to remove cell debris, and added a preservative like sodium azide before being stored at 4° C.

Bio-Plex Procedure

Labelling of Beads with Antibody

Bio-Rad Mag Plex beads (www.bio-rad.com) representing different regions, were labelled with Sigma-Aldrich product M4434 (goat-anti murine IgG2a antibody; www.sigma-adrich.com) using the Bio-Rad bead labelling kit (www.bio-rad.com) as instructed by the manufacturer. Approximately, 5×10⁶ beads were labelled, using 50 μg M4434 for each reaction.

Labelling of Beads with TB-Antigen

Approximately 1-2×10⁶ beads of a specific region were added to 1 ml of a specific TB fusion protein containing growth media, and incubated for 1 hour at room temperature. For fusion proteins produced in low quantities, the beads were repeatedly incubated with more growth media to saturate the beads. The beads were then washed with 1 ml PBS (www.sigma-adrich.com) 3 times and then resuspended in 150 μl PBS per 1×10⁶ TB-antigen loaded beads.

### Preparation of Sample

2 μl of each serum sample to be tested was added to 20 μl PBS or Chaser Buffer.

Incubation of TB-Antigen Labelled Beads with Human Sera

Beads representing different regions with corresponding TB-antigens attached, were mixed in equal amounts and washed once in PBS. The bead mix was resuspended in 150 μl PBS per 1×10⁶ TB-antigen loaded beads. Approximately 100 μl of this mix (1×10⁶) beads was used per 100 samples to be tested, and when testing 5 different antigens. For each 100 samples, 100 μl of the bead mix was added 5.5 ml of PBS with 0.05% Tween 20, 1% skimmed milk, 1% BSA and 60 mM NH₄SCN.

2 μl of each serum sample to be tested was added to 20 μl PBS or Chaser Buffer. The 20 μl PBS containing the 2 μl of serum was then added 50 μl of the bead mix, and the 70 μl reaction mixture was incubated at room temperature, or at 37° C., with shaking in a Bio-Plex incubation tray for 1 hour. The incubation tray was washed by the Bio-Plex magnetic washer, and the washed beads were resuspended in 50 μl of a 5.5 ml PBS solution containing 0.05% Tween 20 and 5.5 μl of anti-human IgG antibody (Sigma-Aldrich product P9170).

Secondary PE-labelled antibody was added and incubated at room temperature, or at 37° C., with shaking in a Bio-Plex incubation tray for 1 hour. The incubation tray was washed using Bio-Plex magnetic washer, and the washed beads were resuspended in 100 μl of PBS and briefly shaken to resuspend the beads.

Detection of Signal in Bio-Plex

The Bio-Plex machine was used as recommended by the manufacturer. The recorded results where exported to Microsoft Excel, and graphically represented by use of Prism/Graphpad.

Sandwich ELISA Procedure

Coating of ELISA Wells with Antibody

10 μg of Sigma-Aldrich product P9170 was added to 10 ml of PBS. 100 μl was added to each well of a 96 well Maxisorb ELISA plate (www.nunc.com). The Maxisorb plate was incubated at 4° C. for 3-4 days or at room temperature for 16-24 h.

Coating ELISA Wells with TB-Antigen

The antibody-coated Maxisorb ELISA plates were washed three times with PBS containing 0.05% Tween 20. 100 μl of growth medium containing TB-fusion protein was added to each well. The 100 μl of growth medium could contain one or a mix of several antigens. The wells were incubated at 37° C. for 1 hour. If the concentration of the TB-antigens in the growth medium was low, the step was repeated.

Incubation of TB-Antigen Labelled ELISA Wells with Human Sera

Excess fusion protein was removed by washing the wells three times with PBS containing 0.05% Tween 20. 2 μl of each serum sample to be tested was added 98 μl PBS with 0.05% Tween 20, 1% skimmed milk, 1% BSA and 60 mM NH₄SCN and added to a well. The sample solutions were incubated in a Maxisorb plate for 1 hour at 37° C. Excess serum proteins were removed by washing the wells three times with PBS containing 0.05% Tween 20. 2 μl of Sigma-Aldrich product A8542 was then added to 10 ml of PBS containing 0.05% Tween 20, and 100 μl of this solution was added to each well. The Maxisorb plate was then incubated for 1 hour at 37° C.

Detection of Signal in ELISA

Excess secondary antibody was removed by washing the wells three times with PBS containing 0.05% Tween 20. The wells were added 100 μl of a buffer containing alkali phosphatase, resulting in a yellow colour appearing in positive wells. The plate was allowed to develop for 10-40 minutes, and the results were recorded by an automatic ELISA plate reader. The recorded results where exported to Microsoft Excel, and graphically represented by use of Prism/Graphpad.

### Example 1

### Detection of Active TB Using the Bio-Plex Procedure

Test Subjects

The Bio-Plex procedure was carried out on the following test groups:
(i) 78 Ethiopian patients with confirmed active TB and 79 healthy HIV-negative Ethiopian community controls. The community controls were people living in the same neighborhood as the patients.
(ii) 132 patients from Tanzania with confirmed active TB and 60 healthy HIV-negative Tanzanian community controls. The community controls were people living in the same neighborhood as the patients.
(iii) 36 Ethiopian patients with confirmed TB, 96 Ethiopian patients with suspected TB, i.e. patients with TB-like symptoms but where no disease was detected using conventional tests, 36 Ethiopian community controls and 23 Norwegian TB-negative persons.

Fusion Proteins

Five fusion proteins were used in the Bio-Plex procedure. The antigenic regions of the five fusion proteins are Rv3881c, Rv0934, Rv1886, Rv3875 and Rv1860. The anchoring region of each fusion protein is a murine IgG2a Fc fragment.

Results

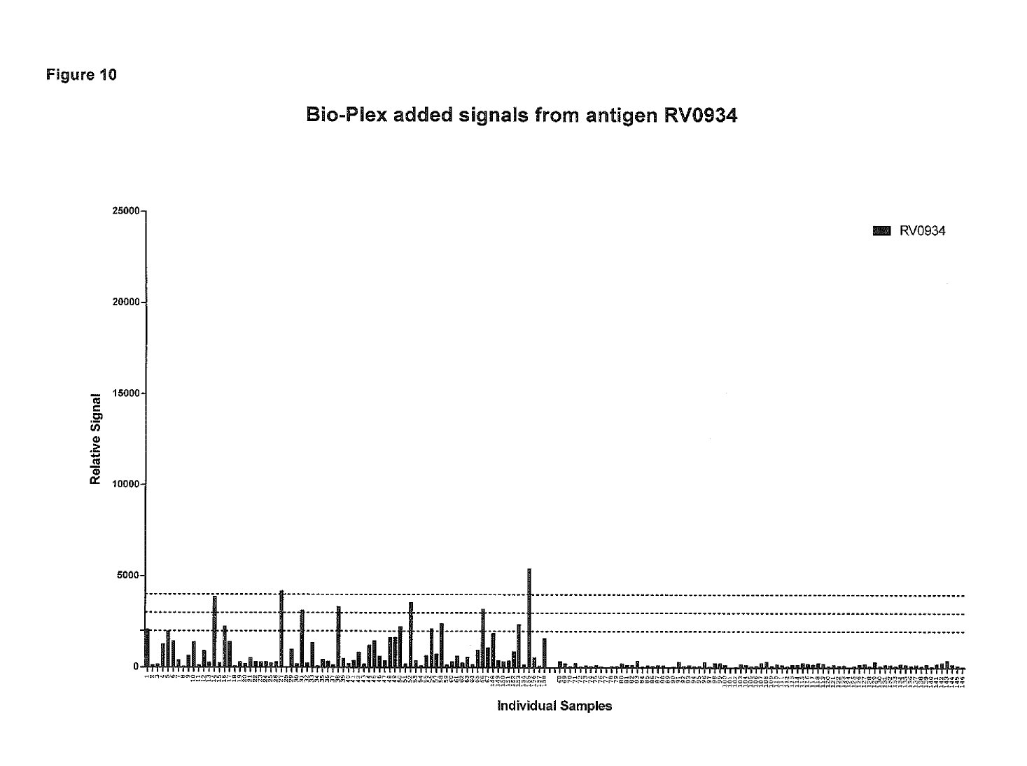

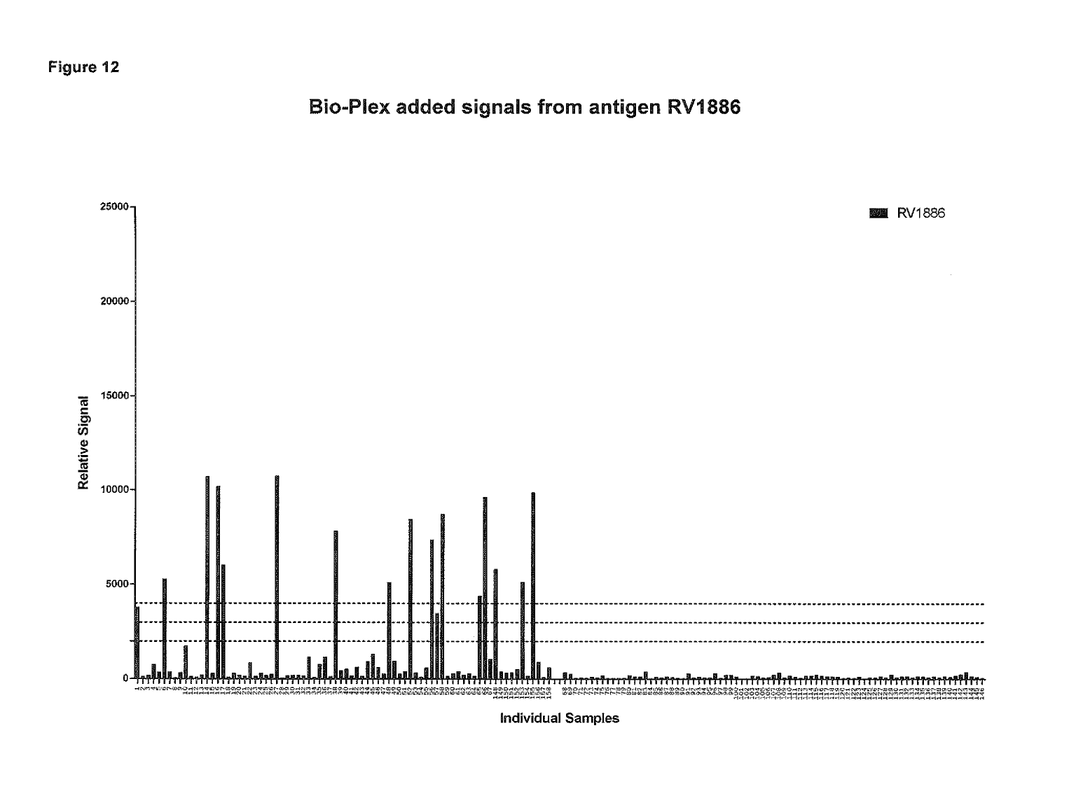

Referring to FIGS. 8(a) and (b), the Bio-Plex test successfully distinguished (50-60%) between patients with active TB and community control patients, i.e. those living in the same neighborhood as the patients with active TB. A sample was defined as positive if the signal generated was at, or above, the predetermined cut off level, i.e. the lowest line in FIG. 8(a) to (c). FIG. 8(a) illustrates the combined signal for all fusion proteins (Rv3881c, Rv0934, Rv1886, Rv3875 and Rv1860) used in the ELISA test. FIGS. 10 to 14, each illustrate the signal obtained from a single fusion protein, i.e., the signal contribution each fusion protein made to the total signal reported in FIG. 8(a).

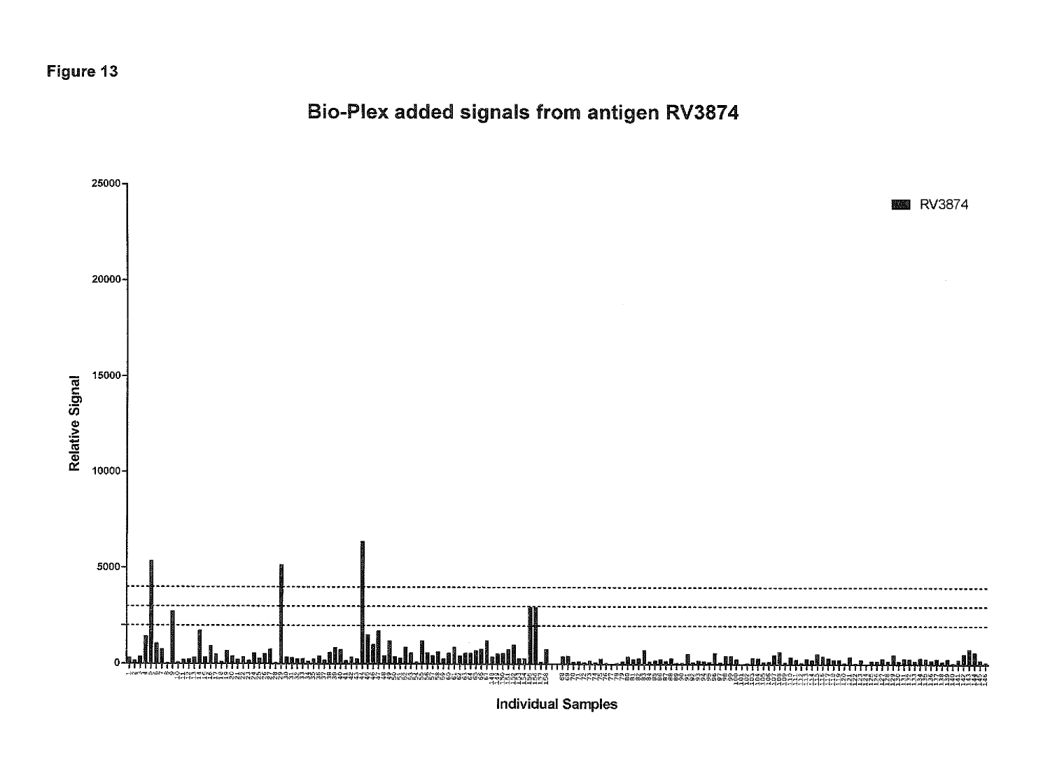

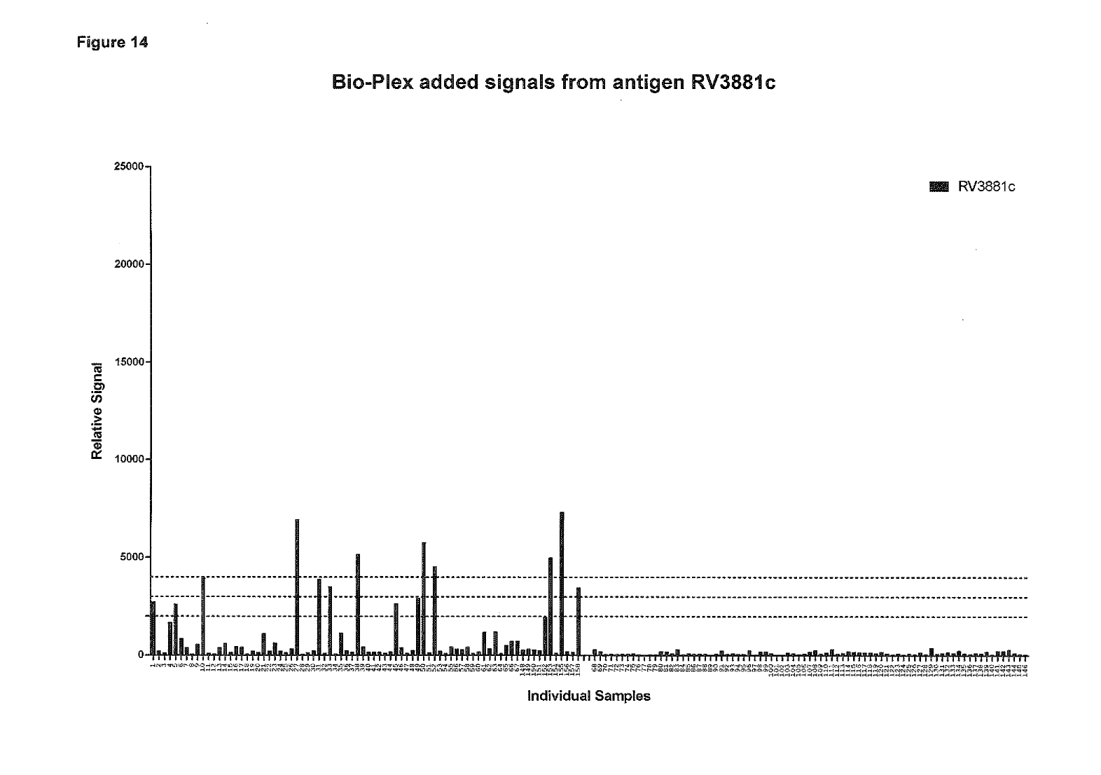

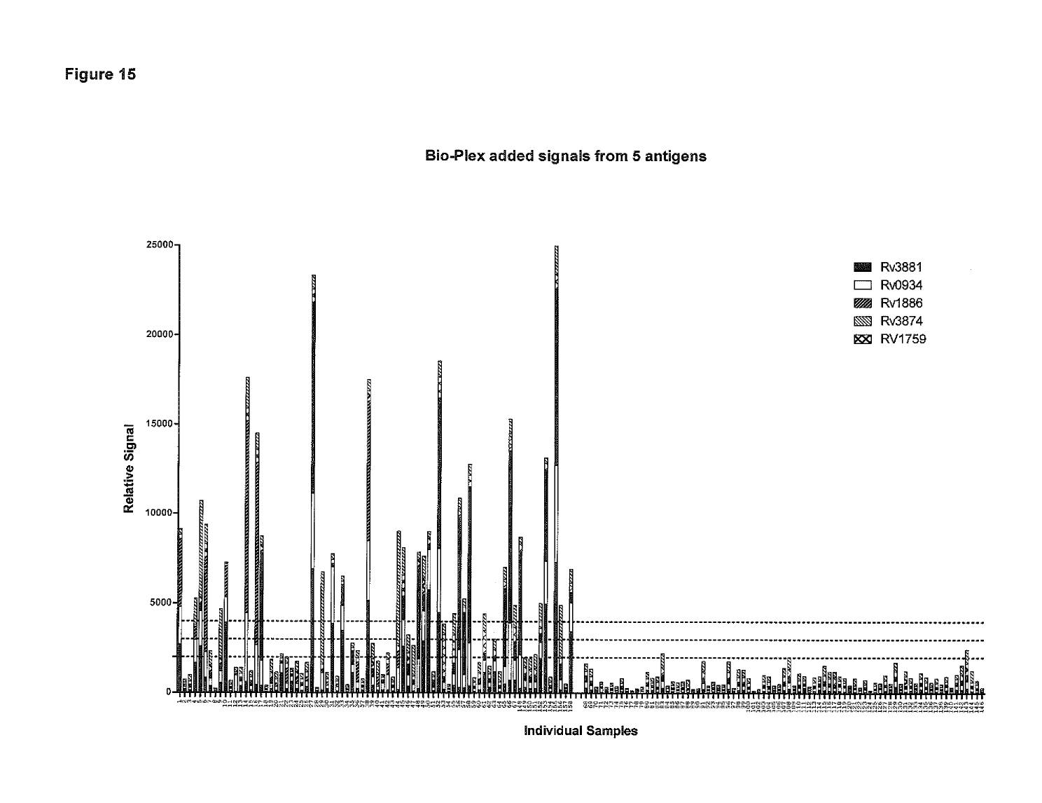

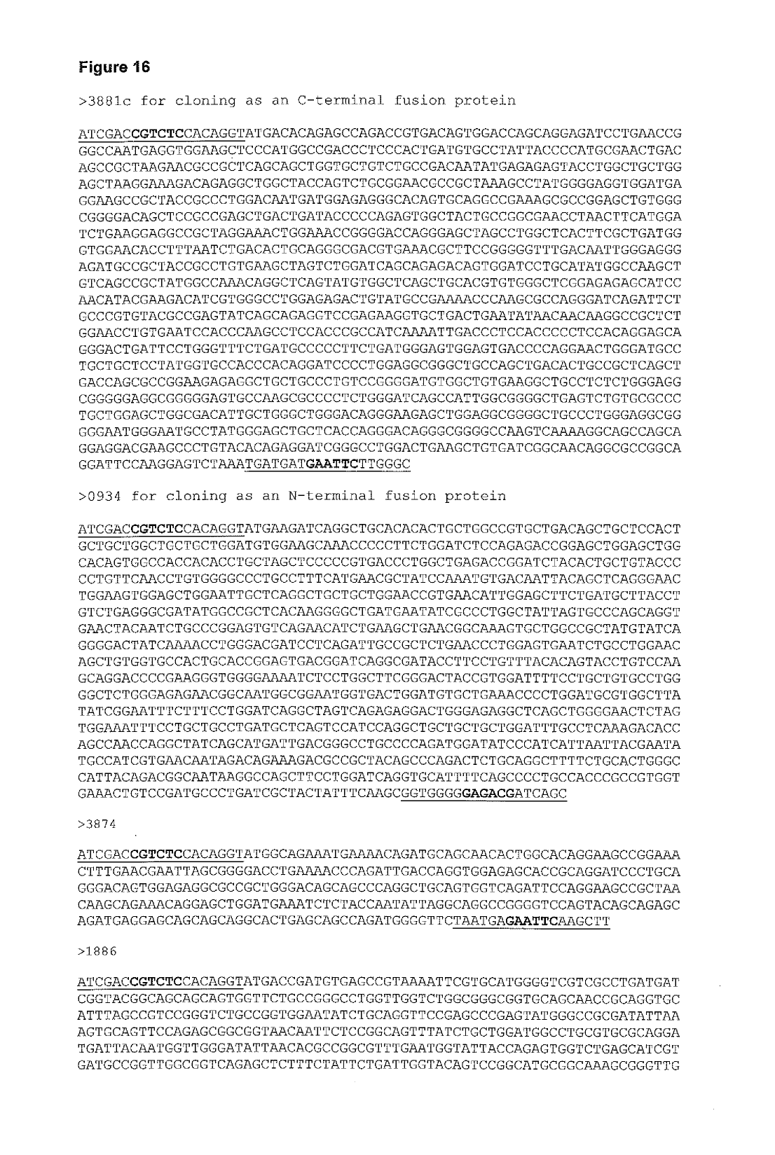

FIG. 10 shows the number of patients testing positive for active TB using Rv1886 fusion protein. FIG. 11 shows the number of patients testing positive for active TB using Rv3874 fusion protein. FIG. 12 illustrates the number of patients testing positive for active TB using Rv1759 fusion protein. FIG. 13 shows the number of patients testing positive for active TB using Rv0934 fusion protein. FIG. 14 shows the number of patients testing positive for active TB using Rv3881c fusion protein.

In FIG. 8(b), 4 patients from the community control group also tested positive for active TB, suggesting that these non-symptomatic persons may be infected and developing TB disease.

Referring to FIG. 8(c) the Bio-Plex test successfully distinguished (50-60%) between patients with active TB and the community controls or the “negative” controls. 4 patients with suspected TB also tested positive for active TB. This result may indicate that the Bio-Plex test detected active TB in a patient where conventional sputum smear microscopy and clinical diagnosis had failed.

Validation

All patients tested were confirmed positive by smear tests and by growing cultures of TB bacteria from sputum samples taken from each patient.

### Example 2

### Detection of Active TB Using the Rapid Test TB “Prototype” ELISA

Test Subjects

The Rapid Test TB “prototype” ELISA was carried out on the following test group:
(i) 78 Ethiopian patients with confirmed active TB and 79 healthy HIV-negative Ethiopian community controls. The community controls were people living in the same neighborhood as the patients.

Fusion Proteins

Five fusion proteins were used in the Rapid Test TB “prototype” ELISA. The antigenic regions of the five fusion proteins are Rv3881c, Rv0934, Rv1886, Rv3875 and Rv1860, respectively. The anchoring region of each fusion protein is a murine IgG2a Fc fragment.

Results

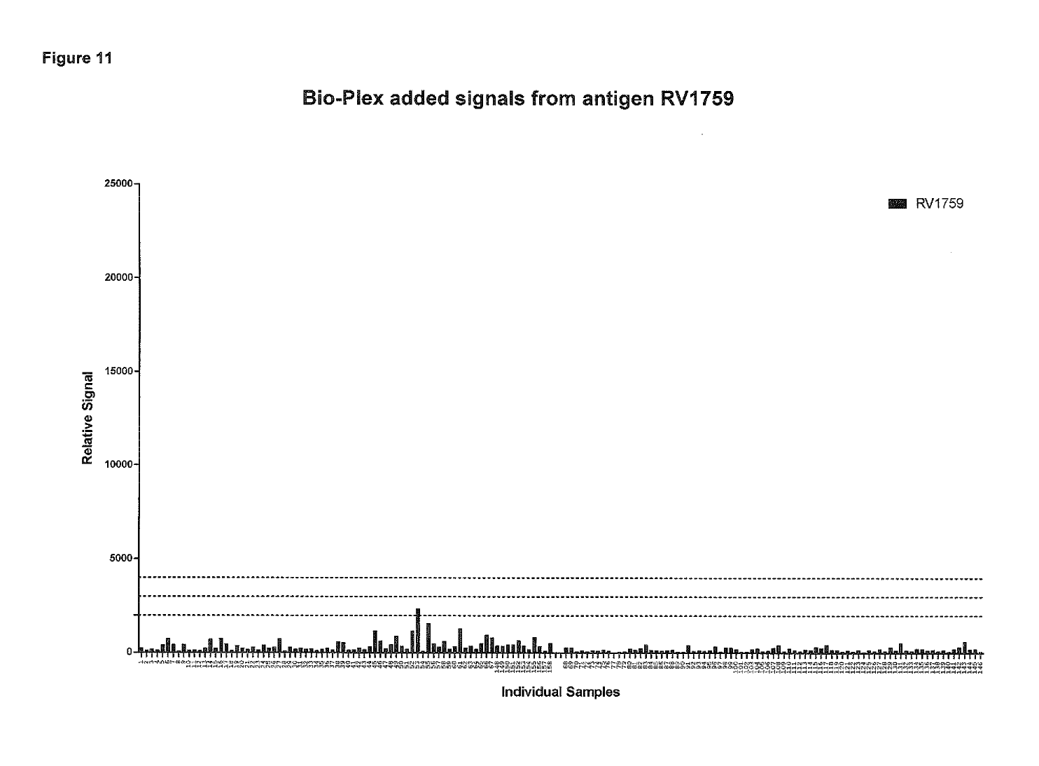

Referring to FIG. 9, the ELISA test successfully distinguished (50-60%) between patients with an active TB and community control patients, i.e. those living in the same neighborhood as the patients with active TB. 2 patients from the community control group also tested positive for active TB, suggesting that these patients are in fact developing a TB disease. A sample was defined as positive if the signal generated was at, or above, the predetermined cut off level, i.e. the lowest line in FIG. 9.

Validation

All patients tested were confirmed positive by smear tests and by growing cultures of TB bacteria from samples taken from each patient.

### Example 3

### Detection of Active TB in a Vietnamese Test Group Using the Bio-Plex Procedure

Test Subjects

The Bio-Plex procedure was carried out on the following test groups:
(i) 62 Vietnamese patients with confirmed active TB and 60 healthy Vietnamese community controls. The community controls were people living in the same neighborhood as the patients.

Antigens

An antigen mix containing three antigens, Rv3881c, Rv0934 and Rv1886, was used to differentiate between patients with active TB and healthy controls.

Results

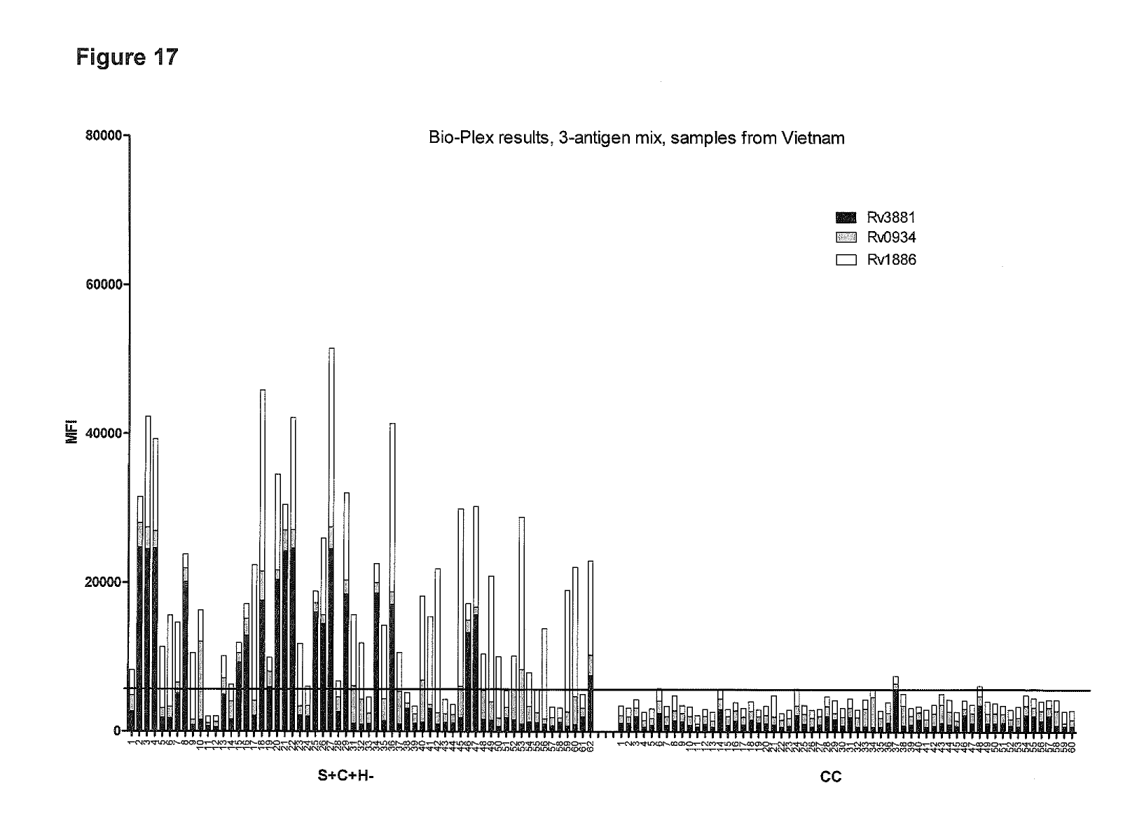

Referring to FIG. 17, the results indicate that a combination of the three antigens has a sensitivity of 83.8% among persons with active TB and a specificity of 96.6% among healthy community controls from Vietnam. Samples from symptomatic persons without TB, i.e. tested negative by traditional smear testing and culture testing, yielded negative results similar to the healthy community controls (12 000 MFI: sensitivity 83.8%, specificity 96.6%). A sample was defined as positive if the signal generated was at, or above, the predetermined cut off level, i.e. the lowest line in FIG. 17.

1-18. (canceled)

19. A method of detecting active TB in a patient comprising the steps of:
(a) providing at least one peptide comprising an amino acid sequence with at least 80% sequence identity to a sequence selected from the group consisting of any one of SEQ. ID NOS. 1 to 3, 8, 4, 7, 6, 5 and an antigenic fragment of one of said sequences;
(b) contacting the at least one peptide with a biological sample obtained from the patient; and
(c) detecting the binding of the at least one peptide with an antibody in the biological sample wherein the presence of an antibody binding to the peptide is indicative of active TB in the patient.

20. The method according to claim 19, wherein the at least one peptide is a plurality of peptides, each comprising a different sequence selected from the group consisting of following (a) to (h):
(a) a sequence with at least 80% sequence identity to SEQ. ID NO. 1 or an antigenic fragment thereof;
(b) a sequence with at least 80% sequence identity to SEQ. ID NO. 2 or an antigenic fragment thereof;
(c) a sequence with at least 80% sequence identity to SEQ. ID NO. 3 or an antigenic fragment thereof;
(d) a sequence with at least 80% sequence identity to SEQ. ID NO. 8 or an antigenic fragment thereof;
(e) a sequence with at least 80% sequence identity to SEQ. ID NO. 4 or an antigenic fragment thereof;
(f) a sequence with at least 80% sequence identity to SEQ. ID NO. 7 or an antigenic fragment thereof;
(g) a sequence with at least 80% sequence identity to SEQ. ID NO. 6 or an antigenic fragment thereof; and
(h) a sequence with at least 80% sequence identity to SEQ. ID NO. 5 or an antigenic fragment thereof.

21. The method according to claim 20, wherein the plurality of peptides comprises at least two of the following:
(a) a sequence with at least 80% sequence identity to a sequence selected from SEQ. ID NOS. 1 or an antigenic fragment thereof;
(b) a sequence with at least 80% sequence identity to a sequence selected from SEQ. ID NOS. 2, or an antigenic fragment thereof; or,
(c) a sequence with at least 80% sequence identity to a sequence selected from SEQ. ID NOS. 3 or an antigenic fragment thereof.

22. The method according to claim 20, wherein the plurality of peptides consists of the following:
(a) a sequence with at least 80% sequence identity to a sequence selected from SEQ. ID NOS. 1 or an antigenic fragment thereof;
(b) a sequence with at least 80% sequence identity to a sequence selected from SEQ. ID NOS. 2, or an antigenic fragment thereof; and,
(c) a sequence with at least 80% sequence identity to a sequence selected from SEQ. ID NOS. 3 or an antigenic fragment thereof.

23. A kit for the detection of active TB in a patient comprising at least two different peptides selected from the group consisting of the following (a) to (h):
(a) a sequence with at least 80% sequence identity to SEQ. ID NO. 1 or an antigenic fragment thereof;
(b) a sequence with at least 80% sequence identity to SEQ. ID NO. 2 or an antigenic fragment thereof;
(c) a sequence with at least 80% sequence identity to SEQ. ID NO. 3 or an antigenic fragment thereof;
(d) a sequence with at least 80% sequence identity to SEQ. ID NO. 8 or an antigenic fragment thereof;
(e) a sequence with at least 80% sequence identity to SEQ. ID NO. 4 or an antigenic fragment thereof;
(f) a sequence with at least 80% sequence identity to SEQ. ID NO. 7 or an antigenic fragment thereof;
(g) a sequence with at least 80% sequence identity to SEQ. ID NO. 6 or an antigenic fragment thereof; and
(h) a sequence with at least 80% sequence identity to SEQ. ID NO. 5 or an antigenic fragment thereof.

24. The kit according to claim 23, comprising;
(a) a peptide comprising an amino acid sequence with at least 80% sequence identity to a sequence selected from SEQ. ID NOS. 1 or an antigenic fragment thereof;
(b) a peptide comprising an amino acid sequence with at least 80% sequence identity to a sequence selected from SEQ. ID NOS. 2, or an antigenic fragment thereof; and,
(c) a peptide comprising an amino acid sequence with at least 80% sequence identity to a sequence selected from SEQ. ID NOS. 3 or an antigenic fragment thereof.

25. A kit for the detection of active TB in a patient comprising:
(a) a solid phase; and
(b) at least one peptide bound to the solid phase wherein the peptide comprises an amino acid sequence with at least 80% sequence identity to a sequence selected from the group consisting of any one of SEQ. ID NOS. 1 to 3, 8, 4, 7, 6, 5 and an antigenic fragment of one of said sequences.

26. The kit according to claim 25, wherein the at least one peptide is a plurality of peptides, each comprising a different sequence selected from the group consisting of the following (a) to (c):
(a) a sequence with at least 80% sequence identity to SEQ. ID NO. 1 or an antigenic fragment thereof;
(b) a sequence with at least 80% sequence identity to SEQ. ID NO. 2 or an antigenic fragment thereof; and
(c) a sequence with at least 80% sequence identity to SEQ. ID NO. 3 or an antigenic fragment thereof.

27. A recombinant fusion protein comprising:
(a) at least one antigenic region comprising an amino acid sequence with at least 80% sequence identity to a sequence selected from the group consisting of any one of SEQ. ID NOS. 1 to 3, 8, 4, 7, 6, 5 and an antigenic fragment of one of said sequences; and
(b) at least one anchoring region comprising a sequence of at least 25 amino acids.

28. The recombinant fusion protein according to claim 27, wherein the at least one antigenic region comprises a plurality of different antigenic regions having a different sequence selected from the group consisting of the following (a) to (h):
(a) a sequence with at least 80% sequence identity to SEQ. ID NO. 1 or an antigenic fragment thereof;
(b) a sequence with at least 80% sequence identity to SEQ. ID NO. 2 or an antigenic fragment thereof;
(c) a sequence with at least 80% sequence identity to SEQ. ID NO. 3 or an antigenic fragment thereof;
(d) a sequence with at least 80% sequence identity to SEQ. ID NO. 8 or an antigenic fragment thereof;
(e) a sequence with at least 80% sequence identity to SEQ. ID NO. 4 or an antigenic fragment thereof;
(f) a sequence with at least 80% sequence identity to SEQ. ID NO. 7 or an antigenic fragment thereof;
(g) a sequence with at least 80% sequence identity to SEQ. ID NO. 6 or an antigenic fragment thereof; and
(h) a sequence with at least 80% sequence identity to SEQ. ID NO. 5 or an antigenic fragment thereof.

29. The recombinant fusion protein according to claim 27, wherein the at least one antigenic region is N-terminal to the at least one anchoring region.

30. The recombinant fusion protein according to claim 27, wherein the at least one antigenic region is C-terminal to the at least one anchoring region.

31. The recombinant fusion protein according to claim 27, wherein the fusion protein is glycosylated.

32. The recombinant fusion protein according to claim 27, wherein the at least one anchoring region comprises at least constant domains 2 and 3 and a hinge region of an immunoglobulin Fc region.

33. The recombinant fusion protein according to claim 32, wherein the immunoglobulin Fc region is a murine IgG2a Fc region.

34. The recombinant fusion protein according to claim 27, wherein the at least one anchoring region comprises an entire IgG antibody.

35. The recombinant fusion protein according to claim 34, wherein the entire IgG antibody is a murine IgG2a antibody.

36. An antigenic region comprising an amino acid sequence with at least 80% sequence identity to a sequence selected from the group consisting of any one of SEQ. ID NOS. 1 to 3, 8, 4, 7, 6, 5 and an antigenic fragment of one of said sequences, wherein the antigenic region is glycosylated.

37. A nucleic acid molecule comprising a nucleotide sequence encoding the recombinant fusion protein according to claim 27.

38. A nucleic acid molecule comprising a nucleotide sequence encoding the antigenic region according to claim 36.

39. A vector comprising the nucleic acid molecule according to claim 37.

40. A vector comprising the nucleic acid molecule according to claim 38.

41. A host cell comprising a nucleic acid molecule according to claim 37, wherein the host cell is capable of expressing a recombinant fusion protein comprising:
(a) at least one antigenic region comprising an amino acid sequence with at least 80% sequence identity to a sequence selected from the group consisting of any one of SEQ. ID NOS. 1 to 3, 8, 4, 7, 6, 5 and an antigenic fragment of one of said sequences; and
(b) at least one anchoring region comprising a sequence of at least 25 amino acids.

42. A host cell comprising a nucleic acid molecule according to claim 38, wherein the host cell is capable of expressing an antigenic region comprising an amino acid sequence with at least 80% sequence identity to a sequence selected from the group consisting of any one of SEQ. ID NOS. 1 to 3, 8, 4, 7, 6, 5 and an antigenic fragment of one of said sequences, wherein the antigenic region is glycosylated.

43. A host cell comprising a vector according to claim 39, wherein the host cell is capable of expressing:
a recombinant fusion protein comprising:
(a) at least one antigenic region comprising an amino acid sequence with at least 80% sequence identity to a sequence selected from the group consisting of any one of SEQ. ID NOS. 1 to 3, 8, 4, 7, 6, 5 and an antigenic fragment of one of said sequences;
and
(b) at least one anchoring region comprising a sequence of at least 25 amino acids; or
an antigenic region comprising an amino acid sequence with at least 80% sequence identity to a sequence selected from the group consisting of any one of SEQ. ID NOS. 1 to 3, 8, 4, 7, 6, 5 and an antigenic fragment of one of said sequences, wherein the antigenic region is glycosylated.

44. A host cell comprising a vector according to claim 40, wherein the host cell is capable of expressing:
a recombinant fusion protein comprising:
(a) at least one antigenic region comprising an amino acid sequence with at least 80% sequence identity to a sequence selected from the group consisting of any one of SEQ. ID NOS. 1 to 3, 8, 4, 7, 6, 5 and an antigenic fragment of one of said sequences;
and
(b) at least one anchoring region comprising a sequence of at least 25 amino acids; or
an antigenic region comprising an amino acid sequence with at least 80% sequence identity to a sequence selected from the group consisting of any one of SEQ. ID NOS. 1 to 3, 8, 4, 7, 6, 5 and an antigenic fragment of one of said sequences, wherein the antigenic region is glycosylated.


SEQUENCE LISTING (28 sequences)

SEQ ID NO: 1 - protein, 460 aa, Mycobacterium tuberculosis
MTQSQTVTVDQQEILNRANEVEAPMADPPTDVPITPCELTAAKNAAQQLVLSADNMREYLAAGAKERQRLATSLRNAAKAYGEVDEEAATALDNDGEGTVQAESAGAVGGDSSAELTDTPRVATAGEPNFMDLKEAARKLETGDQGASLAHFADGWNTFNLTLQGDVKRFRGFDNWEGDAATACEASLDQQRQWILHMAKLSAAMAKQAQYVAQLHVWARREHPTYEDIVGLERLYAENPSARDQILPVYAEYQQRSEKVLTEYNNKAALEPVNPPKPPPAIKIDPPPPPQEQGLIPGFLMPPSDGSGVTPGTGMPAAPMVPPTGSPGGGLPADTAAQLTSAGREAAALSGDVAVKAASLGGGGGGGVPSAPLGSAIGGAESVRPAGAGDIAGLGQGRAGGGAALGGGGMGMPMGAAHQGQGGAKSKGSQQEDEALYTEDRAWTEAVIGNRRRQDSKESK

SEQ ID NO: 2 - protein, 374 aa, Mycobacterium tuberculosis
MKIRLHTLLAVLTAAPLLLAAAGCGSKPPSGSPETGAGAGTVATTPASSPVTLAETGSTLLYPLFNLWGPAFHERYPNVTITAQGTGSGAGIAQAAAGTVNIGASDAYLSEGDMAAHKGLMNIALAISAQQVNYNLPGVSEHLKLNGKVLAAMYQGTIKTWDDPQIAALNPGVNLPGTAVVPLHRSDGSGDTFLFTQYLSKQDPEGWGKSPGFGTTVDFPAVPGALGENGNGGMVTGCAETPGCVAYIGISFLDQASQRGLGEAQLGNSSGNFLLPDAQSIQAAAAGFASKTPANQAISMIDGPAPDGYPIINYEYAIVNNRQKDAATAQTLQAFLHWAITDGNKASFLDQVHFQPLPPAVVKLSDALIATISS

SEQ ID NO: 3 - protein, 325 aa, Mycobacterium tuberculosis
MTDVSRKIRAWGRRLMIGTAAAVVLPGLVGLAGGAATAGAFSRPGLPVEYLQVPSPSMGRDIKVQFQSGGNNSPAVYLLDGLRAQDDYNGWDINTPAFEWYYQSGLSIVMPVGGQSSFYSDWYSPACGKAGCQTYKWETFLTSELPQWLSANRAVKPTGSAAIGLSMAGSSAMILAAYHPQQFIYAGSLSALLDPSQGMGPSLIGLAMGDAGGYKAADMWGPSSDPAWERNDPTQQIPKLVANNTRLWVYCGNGTPNELGGANIPAEFLENFVRSSNLKFQDAYNAAGGHNAVFNFPPNGTHSWEYWGAQLNAMKGDLQSSLGAG

SEQ ID NO: 4 - protein, 884 aa, Mycobacterium tuberculosis
AAAANTTSLLAAGADEISAAIAALFGAHGRAYQAASAEAAAFHGRFVQALTTGGGAYAAAEAAAVTPLLNSINAPVLAATGRPLIGNGANGAPGTGANGGDAGWLIGNGGAGGSGAKGANGGAGGPGGAAGLFGNGGAGGAGGTATANNGIGGAGGAGGSAMLFGAGGAGGAGGAATSLVGGIGGTGGTGGNAGMLAGAAGAGGAGGFSFSTAGGAGGAGGAGGLFTTGGVGGAGGQGHTGGAGGAGGAGGLFGAGGMGGAGGFGDHGTLGTGGAGGDGGGGGLFGAGGDGGAGGSGLTTGGAAGNGGNAGTLSLGAAGGAGGTGGAGGTVFGGGKGGAGGAGGNAGMLFGSGGGGGTGGFGFAAGGQGGVGGSAGMLSGSGGSGGAGGSGGPAGTAAGGAGGAGGAPGLIGNGGNGGNGGESGGTGGVGGAGGNAVLIGNGGEGGIGALAGKSGFGGFGGLLLGADGYNAPESTSPWHNLQQDILSFINEPTEALTGRPLIGNGDSGTPGTGDDGGAGGWLFGNGGNGGAGAAGTNGSAGGAGGAGGILFGTGGAGGAGGVGTAGAGGAGGAGGSAFLIGSGGTGGVGGAATTTGGVGGAGGNAGLLIGAAGLGGCGGGAFTAGVTTGGAGGTGGAAGLFANGGAGGAGGTGSTAGGAGGAGGAGGLYAHGGTGGPGGNGGSTGAGGTGGAGGPGGLYGAGGSGGAGGHGGMAGGGGGVGGNAGSLTLNASGGAGGSGGSSLSGKAGAGGAGGSAGLFYGSGGAGGNGGYSLNGTGGDGGTGGAGQITGLRSGFGGAGGAGGASDTGAGGNGGAGGKAGLYGNGGDGGAGGDGATSGKGGAGGNAVVIGNGGNGGNAGKAGGTAGAGGAGGLVLGRDGQHGLT

SEQ ID NO: 5 - protein, 227 aa, Mycobacterium tuberculosis
MRIKIFMLVTAVVLLCCSGVATAAPKTYCEELKGTDTGQACQIQMSDPAYNINISLPSYYPDQKSLENYIAQTRDKFLSAATSSTPREAPYEYITSATYQSAIPPRGTQAVVLKVYQNAGGTHPTTTYKAFDWDQAYRKPITYDTLWQADTDPLPVVFPIVQGELSKQTGQQVSIAPNAGLDPVNYQNFAVTNDGVIFFFNPGELLPEAAGPTQVLVPRSAIDSMLA

SEQ ID NO: 6 - protein, 325 aa, Mycobacterium tuberculosis
MHQVDPNLTRRKGRLAALAIAAMASASLVTVAVPATANADPEPAPPVPTTAASPPSTAAAPPAPATPVAPPPPAAANTPNAQPGDPNAAPPPADPNAPPPPVIAPNAPQPVRIDNPVGGFSFALPAGWVESDAAHFDYGSALLSKTTGDPPFPGQPPPVANDTRIVLGRLDQKLYASAEATDSKAAARLGSDMGEFYMPYPGTRINQETVSLDANGVSGSASYYEVKFSDPSKPNGQIWTGVIGSPAANAPDAGPPQRWFVVWLGTANNPVDKGAAKALAESIRPLVAPPPAPAPAPAEPAPAPAPAGEVAPTPTTPTPQRTLPA

SEQ ID NO: 7 - protein, 100 aa, Mycobacterium tuberculosis
MAEMKTDAATLAQEAGNFERISGDLKTQIDQVESTAGSLQGQWRGAAGTAAQAAVVRFQEAANKQKQELDEISTNIRQAGVQYSRADEEQQQALSSQMGF

SEQ ID NO: 8 - protein, 96 aa, Mycobacterium tuberculosis
NMTEQQWNFAGIEAAASAIQGNVTSIHSLLDEGKQSLTKLAAAWGGSGSEAYQGVQQKWDATATELNNALQNLARTISEAGQAMASTEGNVTGMFA

SEQ ID NO: 9 - DNA, 1418 bp, Artificial
  Sequence from Mycobacterium tuberculosis artificially optimised for expression in human cells.
atcgaccgtctccacaggtatgacacagagccagaccgtgacagtggaccagcaggagatcctgaaccgggccaatgaggtggaagctcccatggccgaccctcccactgatgtgcctattaccccatgcgaactgacagccgctaagaacgccgctcagcagctggtgctgtctgccgacaatatgagagagtacctggctgctggagctaaggaaagacagaggctggctaccagtctgcggaacgccgctaaagcctatggggaggtggatgaggaagccgctaccgccctggacaatgatggagagggcacagtgcaggccgaaagcgccggagctgtgggcggggacagctccgccgagctgactgatacccccagagtggctactgccggcgaacctaacttcatggatctgaaggaggccgctaggaaactggaaaccggggaccagggagctagcctggctcacttcgctgatgggtggaacacctttaatctgacactgcagggcgacgtgaaacgcttccgggggtttgacaattgggagggagatgccgctaccgcctgtgaagctagtctggatcagcagagacagtggatcctgcatatggccaagctgtcagccgctatggccaaacaggctcagtatgtggctcagctgcacgtgtgggctcggagagagcatccaacatacgaagacatcgtgggcctggagagactgtatgccgaaaacccaagcgccagggatcagattctgcccgtgtacgccgagtatcagcagaggtccgagaaggtgctgactgaatataacaacaaggccgctctggaacctgtgaatccacccaagcctccacccgccatcaaaattgaccctccaccccctccacaggagcagggactgattcctgggtttctgatgcccccttctgatgggagtggagtgaccccaggaactgggatgcctgctgctcctatggtgccacccacaggatcccctggaggcgggctgccagctgacactgccgctcagctgaccagcgccggaagagaggctgctgccctgtccggggatgtggctgtgaaggctgcctctctgggaggcgggggaggcgggggagtgccaagcgcccctctgggatcagccattggcggggctgagtctgtgcgccctgctggagctggcgacattgctgggctgggacagggaagagctggaggcggggctgccctgggaggcgggggaatgggaatgcctatgggagctgctcaccagggacagggcggggccaagtcaaaaggcagccagcaggaggacgaagccctgtacacagaggatcgggcctggactgaagctgtgatcggcaacaggcgccggcaggattccaaggagtctaaatgatgatgaattcttgggc

SEQ ID NO: 10 - protein, 460 aa, Artificial; identical to SEQ ID NO: 1
  Synthetic Construct

SEQ ID NO: 11 - DNA, 1160 bp, artificial
  Rv0934 sequence from Mycobacterium tuberculosis artificially optimised for expression in human cells
atcgaccgtctccacaggtatgaagatcaggctgcacacactgctggccgtgctgacagctgctccactgctgctggctgctgctggatgtggaagcaaacccccttctggatctccagagaccggagctggagctggcacagtggccaccacacctgctagctcccccgtgaccctggctgagaccggatctacactgctgtaccccctgttcaacctgtggggccctgcctttcatgaacgctatccaaatgtgacaattacagctcagggaactggaagtggagctggaattgctcaggctgctgctggaaccgtgaacattggagcttctgatgcttacctgtctgagggcgatatggccgctcacaaggggctgatgaatatcgccctggctattagtgcccagcaggtgaactacaatctgcccggagtgtcagaacatctgaagctgaacggcaaagtgctggccgctatgtatcaggggactatcaaaacctgggacgatcctcagattgccgctctgaaccctggagtgaatctgcctggaacagctgtggtgccactgcaccggagtgacggatcaggcgataccttcctgtttacacagtacctgtccaagcaggaccccgaagggtggggaaaatctcctggcttcgggactaccgtggattttcctgctgtgcctggggctctgggagagaacggcaatggcggaatggtgactggatgtgctgaaacccctggatgcgtggcttatatcggaatttctttcctggatcaggctagtcagagaggactgggagaggctcagctggggaactctagtggaaatttcctgctgcctgatgctcagtccatccaggctgctgctgctggatttgcctcaaagacaccagccaaccaggctatcagcatgattgacgggcctgccccagatggatatcccatcattaattacgaatatgccatcgtgaacaatagacagaaagacgccgctacagcccagactctgcaggcttttctgcactgggccattacagacggcaataaggccagcttcctggatcaggtgcattttcagcccctgccacccgccgtggtgaaactgtccgatgccctgatcgctactatttcaagcggtgggggagacgatcagc

SEQ ID NO: 12 - protein, 374 aa, artificial; identical to SEQ ID NO: 2
  Synthetic Construct

SEQ ID NO: 13 - DNA, 1013 bp, artificial
  Rv1886c sequence from Mycobacterium tuberculosis artificially optimised for expression in human cells
atcgaccgtctccacaggtatgaccgatgtgagccgtaaaattcgtgcatggggtcgtcgcctgatgatcggtacggcagcagcagtggttctgccgggcctggttggtctggcgggcggtgcagcaaccgcaggtgcatttagccgtccgggtctgccggtggaatatctgcaggttccgagcccgagtatgggccgcgatattaaagtgcagttccagagcggcggtaacaattctccggcagtttatctgctggatggcctgcgtgcgcaggatgattacaatggttgggatattaacacgccggcgtttgaatggtattaccagagtggtctgagcatcgtgatgccggttggcggtcagagctctttctattctgattggtacagtccggcatgcggcaaagcgggttgtcagacctataaatgggaaacctttctgacgagcgaactgccgcagtggctgtctgcgaatcgtgcagtgaaaccgacgggtagtgcagcaattggtctgagcatggccggcagtagcgcaatgattctggccgcatatcatccgcagcagttcatctacgcaggtagcctgtctgcactgctggatccgagccagggcatgggtccgagtctgatcggtctggcgatgggtgatgccggcggttataaagcggccgatatgtggggtccgtctagtgatccggcctgggaacgtaacgatccgacccagcagattccgaaactggtggcgaacaatacccgcctgtgggtttactgcggcaacggtacgccgaatgaactgggcggtgcaaatatcccggcggaatttctggaaaatttcgtgcgtagctctaacctgaaatttcaggatgcctataacgcagcgggcggtcataacgcagtttttaatttcccgccgaacggtacccacagctgggaatactggggcgcccagctgaacgcaatgaaaggtgatctgcagagtagcctgggcgcgggtggtgggggagacgatcagc

SEQ ID NO: 14 - protein, 325 aa, artificial; identical to SEQ ID NO: 3
  Synthetic Construct

SEQ ID NO: 15 - DNA, 2686 bp, Artificial Sequence
  Rv1759c sequence from Mycobacterium tuberculosis artificially optimised for expression in human cells
aaccgtctccacaggtgccgctgcagccaacaccacaagtctgctggctgcaggcgccgatgagatctcagccgctattgcagcactgttcggagctcacggacgagcataccaggctgcatctgccgaagccgctgcattccatggcagatttgtgcaggccctgactaccggaggaggagcttatgcagctgcagaggccgctgcagtgactccactgctgaacagcatcaatgcaccagtcctggccgctaccggacgacctctgattggcaacggggctaatggagcaccaggaacaggagcaaacggaggggacgccggatggctgatcggaaatggaggagcaggaggatccggagcaaaaggagctaacggcggagcaggaggaccaggaggagcagcaggcctgttcgggaacggcggggcaggaggcgccgggggaacagccactgctaacaatggaatcggaggagccggcggagcaggaggatctgccatgctgtttggagcaggaggagctggaggagcaggaggagctgcaaccagtctggtgggaggaattggaggaaccggaggaacaggaggaaatgcaggaatgctggctggagcagccggcgcaggaggagctgggggattctcttttagtactgcaggcggcgccggcggcgccggaggagcaggagggctgttcacaactggaggagtcggaggagcaggaggacagggacacaccggaggcgccgggggagctggcggggcaggaggcctgttcggagcaggaggaatgggaggagctggaggatttggggaccatggaactctgggcaccgggggagccggcggggatggaggcgggggaggcctgtttggagcaggaggcgacggcggagctggaggatcaggactgaccacaggaggagcagcaggaaacggaggaaatgcaggaaccctgagcctgggcgctgcaggaggagcaggaggaaccggaggagctggaggaacagtgttcggcggcggcaaaggcggcgccggcggagctggaggcaacgccggaatgctgtttggaagcggaggaggaggaggaacaggaggattcggatttgcagctggaggacagggaggagtcggaggatccgcaggaatgctgtcaggaagcggaggcagtgggggagccggcgggtcaggaggccctgctggcacagcagccgggggagccgggggagcaggaggagctccagggctgatcggaaacgggggaaatggcgggaacggaggcgagtctggaggaactggaggagtgggaggagcaggaggaaacgcagtcctgattggcaatggaggagaaggaggaattggagcactggccggcaagagtggattcggaggatttggaggactgctgctgggagctgatggctacaacgcacccgaatccacctctccttggcacaatctgcagcaggacatcctgtccttcattaacgagccaactgaagccctgaccggcagacccctgatcgggaatggagattctggcacacctggaactggggacgatggcggcgcaggaggatggctgtttggcaatggaggaaacggaggagcaggagctgcaggaactaacggaagcgctggcggcgccgggggagctggaggcattctgttcggaacaggaggagccggaggcgcaggaggagtgggaactgctggagcagggggcgcaggcggcgcaggaggaagcgcctttctgatcggctccggaggaaccggcggcgtgggaggagcagctactaccacaggaggagtcggaggagctggaggaaatgcaggactgctgattggagcagcaggactgggaggatgcggaggaggagccttcaccgctggcgtgactaccggaggagcaggaggaacaggaggagctgcaggcctgtttgccaacgggggcgccggcggcgcaggaggaaccggaagcaccgccggaggcgccggcggagctgggggagctggcgggctgtacgcccacggaggcacagggggacccggcgggaacggaggcagtacaggagcaggaggaactggaggagctggaggacctgggggactgtatggcgctggaggctcaggaggcgctgggggacatggaggaatggccggaggaggaggaggagtgggaggaaacgcaggctctctgaccctgaatgccagtggaggcgccggaggatcaggaggaagctccctgagcggaaaggccggcgcaggcggggcaggaggatccgcaggactgttctacggatctggcggcgcaggaggaaacggaggatatagcctgaatggcactggaggagatggcggaaccggaggagcaggacagatcacaggactgaggagcggcttcggcggagccggaggggcaggaggagcttccgacactggagcaggaggaaatggaggagctggaggcaaggcagggctgtatggaaacggaggcgacggcggagcaggaggcgatggcgctacctccggcaaaggcggggcaggaggaaacgctgtggtcattggcaatggaggcaacggaggaaatgcaggcaaggcaggaggaaccgcaggagcaggaggagccgggggactggtgctgggacgcgacgggcagcatggactgacataatgaaagcttgaattc

SEQ ID NO: 16 - protein, 884 aa, Artificial Sequence; identical to SEQ ID NO: 4
  Synthetic Construct

SEQ ID NO: 17 - DNA, 719 bp, artificial
  Rv1980c sequence from Mycobacterium tuberculosis artificially optimised for expression in human cells
atcgaccgtctccacaggtatgcggatcaagatcttcatgctggtgacagccgtggtgctgctgtgctgttctggcgtggctactgccgctcccaagacctactgcgaggaactgaaaggaaccgacacaggccaggcctgtcagatccagatgtccgatcctgcttacaacatcaatatttctctgcctagttactatccagaccagaagagcctggagaactatattgcccagacccgggataaattcctgtccgccgctactagctccacccccagagaggccccttacgaatatatcacatccgccacttatcagtctgctattccccctaggggaacacaggctgtggtgctgaaggtgtaccagaatgctggcgggactcaccctaccacaacttataaggcctttgactgggatcaggcttaccgcaaaccaatcacctatgacacactgtggcaggctgacacagatccactgcctgtggtgttcccaatcgtgcagggggaactgagtaaacagacaggacagcaggtgtcaattgccccaaacgctgggctggaccccgtgaactaccagaatttcgccgtgactaacgatggcgtgatcttctttttcaatcctggagagctgctgcctgaagctgctggacctacccaggtgctggtgccaaggtcagccattgatagcatgctggctggtgggggagacgatcagc

SEQ ID NO: 18 - protein, 227 aa, artificial; identical to SEQ ID NO: 5
  Synthetic Construct

SEQ ID NO: 19 - DNA, 1013 bp, artificial
  Rv1860 sequence from Mycobacterium tuberculosis artificially optimised for expression in human cells
atcgaccgtctccacaggtatgcaccaggtggaccccaacctgacccggagaaaaggccggctggccgctctggctattgctgctatggctagcgcctccctggtgactgtggctgtgccagctaccgccaatgctgatcccgaaccagcccctcccgtgccaaccacagctgctagcccaccctccaccgccgctgcccctccagccccagctacacctgtggcccctcccccacccgctgctgctaacacacctaatgctcagccaggggacccaaatgctgctcctccacccgctgatcccaatgcccctccaccccctgtgatcgctcctaacgccccacagcccgtgcggattgacaatcctgtgggcgggttcagttttgctctgcctgctggatgggtggaatcagatgctgctcatttcgattacgggtcagccctgctgagcaaaactaccggagatccacccttccctggacagcctccacccgtggctaatgacactaggatcgtgctggggcgcctggatcagaagctgtatgcctccgctgaggccaccgactctaaagctgctgctcggctgggaagtgatatgggagagttctacatgccttatccaggaacaagaattaaccaggagaccgtgagcctggacgctaatggagtgtctggcagtgcctcatactatgaagtgaagttttctgatcccagtaaacctaacggacagatctggacaggcgtgatcggatctcctgctgctaacgctcctgatgctggccctccacagaggtggttcgtggtgtggctggggactgccaacaatcctgtggataagggagctgccaaagctctggctgaatccatcagaccactggtggcccctcccccagctcctgctcctgctccagccgagcccgctcctgccccagctcccgccggcgaagtggcccctaccccaacaactcccacccctcagcggacactgcccgccggtgggggagacgatcagc

SEQ ID NO: 20 - protein, 325 aa, artificial; identical to SEQ ID NO: 6
  Synthetic Construct

SEQ ID NO: 21 - DNA, 337 bp, artificial
  Rv3874 sequence from Mycobacterium tuberculosis artificially optimised for expression in human cells
atcgaccgtctccacaggtatggcagaaatgaaaacagatgcagcaacactggcacaggaagccggaaactttgaacgaattagcggggacctgaaaacccagattgaccaggtggagagcaccgcaggatccctgcagggacagtggagaggcgccgctgggacagcagcccaggctgcagtggtcagattccaggaagccgctaacaagcagaaacaggagctggatgaaatctctaccaatattaggcaggccggggtccagtacagcagagcagatgaggagcagcagcaggcactgagcagccagatggggttctaatgagaattcaagctt

SEQ ID NO: 22 - protein, 100 aa, artificial; identical to SEQ ID NO: 7
  Synthetic Construct

SEQ ID NO: 23 - DNA, 321 bp, artificial
  Rv3875 sequence from Mycobacterium tuberculosis artificially optimised for expression in human cells
atcgaccgtctccacaggtatgaccgaacagcagtggaactttgcgggcattgaagcggccgcaagcgccatccagggtaatgtgaccagtattcatagcctgctggatgaaggcaaacagtctctgacgaaactggcagcagcatggggtggtagcggttctgaagcgtatcagggtgttcagcagaaatgggatgcgaccgccacggaactgaacaatgccctgcagaacctggcacgtacgatttctgaagccggtcaggcaatggcgagtaccgaaggcaatgtgacgggcatgttcgccggtgggggagacgatca

SEQ ID NO: 24 - protein, 95 aa, artificial
  Synthetic Construct
MTEQQWNFAGIEAAASAIQGNVTSIHSLLDEGKQSLTKLAAAWGGSGSEAYQGVQQKWDATATELNNALQNLARTISEAGQAMASTEGNVTGMFA

SEQ ID NO: 25 - protein, 24 aa, Homo sapiens
MAVMAPRTLVLLLSGALALTQTWA

SEQ ID NO: 26 - protein, 17 aa, Mus musculus
TMGWSCIILFLVATATG

SEQ ID NO: 27 - DNA, 9234 bp, Artificial Sequence
  pFRIDA-mlgG2a-N N-terminal cloning vector
gacggatcgggagatccaggaaaaggacaagcagcgaaaattcacgcccccttgggaggtggcggcatatgcaaaggatagcactcccactctactactgggtatcatatgctgactgtatatgcatgaggatagcatatgctacccggatacagattaggatagcatatactacccagatatagattaggatagcatatgctacccagatatagattaggatagcctatgctacccagatataaattaggatagcatatactacccagatatagattaggatagcatatgctacccagatatagattaggatagcctatgctacccagatatagattaggatagcatatgctacccagatatagattaggatagcatatgctatccagatatttgggtagtatatgctacccagatataaattaggatagcatatactaccctaatctctattaggatagcatatgctacccggatacagattaggatagcatatactacccagatatagattaggatagcatatgctacccagatatagattaggatagcctatgctacccagatataaattaggatagcatatactacccagatatagattaggatagcatatgctacccagatatagattaggatagcctatgctacccagatatagattaggatagcatatgctatccagatatttgggtagtatatgctacccatggcaacattagcccaccgtgctctcagcgacctcgtgaatatgaggaccaacaaccctgtgcttggcgctcaggcgcaagtgtgtgtaatttgtcctccagatcgcagcaatcgcgcccctatcttggcccgcccacctacttatgcaggtattccccggggtgccattagtggttttgtgggcaagtggtttgaccgcagtggttagcggggttacaatcagccaagttattacacccttattttacagtccaaaaccgcagggcggcgtgtgggggctgacgcgtgcccccactccacaatttcaaaaaaaagagtggccacttgtctttgtttatgggccccattggcgtggagccccgtttaattttcgggggtgttagagacaaccagtggagtccgctgctgtcggcgtccactctctttccccttgttacaaatagagtgtaacaacatggttcacctgtcttggtccctgcctgggacacatcttaataaccccagtatcatattgcactaggattatgtgttgcccatagccataaattcgtgtgagatggacatccagtctttacggcttgtccccaccccatggatttctattgttaaagatattcagaatgtttcattcctacactagtatttattgcccaaggggtttgtgagggttatattggtgtcatagcacaatgccaccactgaaccccccgtccaaattttattctgggggcgtcacctgaaaccttgttttcgagcacctcacatacaccttactgttcacaactcagcagttattctattagctaaacgaaggagaatgaagaagcaggcgaagattcaggagagttcactgcccgctccttgatcttcagccactgcccttgtgactaaaatggttcactaccctcgtggaatcctgaccccatgtaaataaaaccgtgacagctcatggggtgggagatatcgctgttccttaggacccttttactaaccctaattcgatagcatatgcttcccgttgggtaacatatgctattgaattagggttagtctggatagtatatactactacccgggaagcatatgctacccgtttagggttaacaagggggccttataaacactattgctaatgccctcttgagggtccgcttatcggtagctacacaggcccctctgattgacgttggtgtagcctcccgtagtcttcctgggcccctgggaggtacatgtcccccagcatagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagctgtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctagcttggacctcaccatgggatggagctgtatcatcctcttcttggtagcaacagctacagggagacgggcgcgccggatcccgtctcctggggagcccagagggcccacaatcaagccctgtcctccatgcaaatgcccagcacctaacctcttgggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaatgatgagaattcgatatcaagcttgtcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaaacctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacctcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccagggccagggatcagatatccactgacctttggatggtgctacaagctagtaccagttgagcaagagaaggtagaagaagccaatgaaggagagaacacccgcttgttacaccctgtgagcctgcatgggatggatgacccggagagagaagtattagagtggaggtttgacagccgcctagcatttcatcacatggcccgagagctgcatccggatctgaggggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcaaattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 28 - DNA, 9273 bp, Artificial Sequence
  pFRIDA-mlg2a-C C-terminal cloning vector
gacggatcgggagatccaggaaaaggacaagcagcgaaaattcacgcccccttgggaggtggcggcatatgcaaaggatagcactcccactctactactgggtatcatatgctgactgtatatgcatgaggatagcatatgctacccggatacagattaggatagcatatactacccagatatagattaggatagcatatgctacccagatatagattaggatagcctatgctacccagatataaattaggatagcatatactacccagatatagattaggatagcatatgctacccagatatagattaggatagcctatgctacccagatatagattaggatagcatatgctacccagatatagattaggatagcatatgctatccagatatttgggtagtatatgctacccagatataaattaggatagcatatactaccctaatctctattaggatagcatatgctacccggatacagattaggatagcatatactacccagatatagattaggatagcatatgctacccagatatagattaggatagcctatgctacccagatataaattaggatagcatatactacccagatatagattaggatagcatatgctacccagatatagattaggatagcctatgctacccagatatagattaggatagcatatgctatccagatatttgggtagtatatgctacccatggcaacattagcccaccgtgctctcagcgacctcgtgaatatgaggaccaacaaccctgtgcttggcgctcaggcgcaagtgtgtgtaatttgtcctccagatcgcagcaatcgcgcccctatcttggcccgcccacctacttatgcaggtattccccggggtgccattagtggttttgtgggcaagtggtttgaccgcagtggttagcggggttacaatcagccaagttattacacccttattttacagtccaaaaccgcagggcggcgtgtgggggctgacgcgtgcccccactccacaatttcaaaaaaaagagtggccacttgtctttgtttatgggccccattggcgtggagccccgtttaattttcgggggtgttagagacaaccagtggagtccgctgctgtcggcgtccactctctttccccttgttacaaatagagtgtaacaacatggttcacctgtcttggtccctgcctgggacacatcttaataaccccagtatcatattgcactaggattatgtgttgcccatagccataaattcgtgtgagatggacatccagtctttacggcttgtccccaccccatggatttctattgttaaagatattcagaatgtttcattcctacactagtatttattgcccaaggggtttgtgagggttatattggtgtcatagcacaatgccaccactgaaccccccgtccaaattttattctgggggcgtcacctgaaaccttgttttcgagcacctcacatacaccttactgttcacaactcagcagttattctattagctaaacgaaggagaatgaagaagcaggcgaagattcaggagagttcactgcccgctccttgatcttcagccactgcccttgtgactaaaatggttcactaccctcgtggaatcctgaccccatgtaaataaaaccgtgacagctcatggggtgggagatatcgctgttccttaggacccttttactaaccctaattcgatagcatatgcttcccgttgggtaacatatgctattgaattagggttagtctggatagtatatactactacccgggaagcatatgctacccgtttagggttaacaagggggccttataaacactattgctaatgccctcttgagggtccgcttatcggtagctacacaggcccctctgattgacgttggtgtagcctcccgtagtcttcctgggcccctgggaggtacatgtcccccagcatagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagctgtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctagcttggacctcaccatgggatggagctgtatcatcctcttcttggtagcaacagctacaggtgagcccagagggcccacaatcaagccctgtcctccatgcaaatgcccagcacctaacctcttgggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaagacagcgagacgtttaaaccgagggcgctagtgtttaaacggccgccgtctccccccactagtaacggccgccaccgaattcgatatcaagcttgtcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaaacctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacctcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccagggccagggatcagatatccactgacctttggatggtgctacaagctagtaccagttgagcaagagaaggtagaagaagccaatgaaggagagaacacccgcttgttacaccctgtgagcctgcatgggatggatgacccggagagagaagtattagagtggaggtttgacagccgcctagcatttcatcacatggcccgagagctgcatccggatctgaggggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcaaattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc